(19) United States
(12) Patent Application Publication
(10) Pub. No.: US 2026/0022413 A1
(43) Pub. Date: Jan. 22, 2026

(54) ENGINEERING DYNAMIC DNA NANO-DEVICES TO AMPLIFY SIGNAL

(71) Applicant: PRESIDENT AND FELLOWS OF HARVARD COLLEGE, Cambridge, MA (US)

(72) Inventors: Xiaokang LUN, Cambridge, MA (US); Kuanwei SHENG, Cambridge, MA (US); Peng YIN, Cambridge, MA (US)

(73) Assignee: PRESIDENT AND FELLOWS OF HARVARD COLLEGE, Cambridge, MA (US)

(21) Appl. No.: 18/995,018

(22) PCT Filed: Jul. 20, 2023
(86) PCT No.: PCT/US23/28207

§ 371 Date: Jan. 15, 2025

Related U.S. Application Data
(60) Provisional application No. 63/390,831, filed on Jul. 20, 2022.
(60) Provisional application No. 63/390,946, filed on Jul. 20, 2022.

(51) Int. Cl. C12Q 1/6816; C12Q 1/6804; C12Q 1/6841

(52) U.S. Cl. CPC C12Q 1/6816; C12Q 1/6804; C12Q 1/6841

(57) ABSTRACT

The methods, compositions and kits described herein provide signal amplification approaches for the detection of target biomolecules that significantly increase the sensitivity of detection using target-binding ligand molecules. The methods include cyclic addition of nucleic acid repeats to, e.g., target-binding molecules, thereby providing multiple landing pads for labeled probes. The methods are well-suited to performance in multiplex, thereby permitting the sensitive detection of multiple targets in a single assay. These methods and compositions can be applied to, among other things, imaging, flow cytometry, and mass cytometry/Cy TOF. providing additional tools for research and diagnostic purposes.

### CROSS REFERENCE TO RELATED APPLICATIONS

This application is a 371 National Phase Entry of International Patent Application No. PCT/US23/28207, filed Jul. 20, 2023, which claims benefit under 35 U.S.C. § 119(e) of U.S. Provisional Application No. 63/390,831, filed Jul. 20, 2022 and U.S. Provisional Application No. 63/390,946, filed Jul. 20, 2022, the contents of each of which are incorporated herein by reference in their entireties.

### GOVERNMENT SUPPORT

This invention was made with government support under GM133052 awarded by National Institutes of Health (NIH). The government has certain rights in this invention.

### TECHNICAL FIELD

The technology described herein relates to methods for amplifying signal in detecting, quantifying, and imaging biomolecules.

### BACKGROUND

Methods for detecting, quantitating and/or imaging biological target molecules of interest are central to a wide range of research and diagnostic approaches. There is a need in the art for compositions and methods permitting more sensitive detection of target biomolecules, as well as, for example, detection of a plurality of such target biomolecule in the same assay—so called multiplex detection.

Recently emerging single-cell technologies have allowed investigating mechanisms underlying cancer initiation and progression from a totally new perspective. However, current epitope-based single cell analysis suffers from the inability to incorporate a necessary number of targets of interest in one analysis, while single-cell proteomics methods are limited by sensitivity and throughput. For example, flow cytometry (e.g., BD Fortessa) is limited by the fluorescence spectrum overlapping, and cell autofluorescence. Sequencing-based epitope-quantification approaches, such as CITE-seq and REAP-seq suffer from high technical variance and nonspecific binding introduced by the long oligo barcode, such that they are less quantitative than flow and mass cytometry methods. Lab-on-chip technologies, such as single-cell barcode chips (SCBCs) and single-cell western blotting (scWesterns), are more sensitive than cytometric methods and allow detection of low-abundance proteins. However, the multiplexity and throughput for this type of method are both low. SCoPE-MS and SCoPE2 are emerging single-cell proteomics analyses that have allowed proteome-scale characterization at single-cell level. However, the sensitivity, throughput and reproducibility have to be increased before such types of methods can be used to quantitatively profile protein abundance variance at single-cell level.

Mass cytometry, a recently established approach based on inductively coupled plasma time-of-flight mass spectrometry and a single-cell sample introduction system, allows simultaneous quantification of>50 proteins or protein modifications at single-cell resolution, permitting the profiling of complex cellular behaviors in highly heterogeneous samples. In mass cytometry, metal isotope-tagged antibodies are applied to label proteins or protein modifications in cells. During the sample acquisition, each stained single cell is vaporized, atomized, and ionized. The metals in the formed ion clouds are quantitatively analyzed by a mass spectrometer to yield a high-dimensional single-cell proteomic readout. However, mass cytometry analysis faces a sensitivity limitation requiring the accumulation of˜300 metal-tagged antibodies per epitope to produce a detectable signal. This inherent limitation prevents the use of mass cytometry in profiling the low-abundance proteome, including many transcriptional factors, surface receptor proteins, and intracellular phosphorylation sites that are critical in health and disease. Analyzing cells of small volume, such as immune cells and microbes is even more challenging on a mass cytometer. Systematic profiling of signaling networks in T lymphocytes (a T cell is 23 times smaller than a HeLa cell on average) has not been achieved at single-cell resolution. The abundances of many key phosphorylated proteins in human T lymphocytes do not reach the detection limit of a mass cytometer.

### SUMMARY

The technology described herein relates, in general, to the amplification of signal relating to the binding of a target-binding ligand to its target in or on a cell. Such amplification of signal can provide a read-out with sensitivity and resolution to as much as single-molecule detection. In various embodiments, the technology relates to the generation of concatemers of nucleic acid sequence on a target ligand-binding molecule in which the repeated sequences provide hybridization sites for a plurality of labeled nucleic acid probes per target-binding ligand molecule, thereby amplifying the signal related to the binding of target molecules in a cell sample by the target ligand-binding molecule. The methods, compositions and kits described herein find application in a number of scenarios, but have particular relevance in mass cytometry and fluorescence-activated cell sorting or flow cytometry as described further herein.

In one aspect, described herein is a method of detecting and/or quantitating a target molecule at a single cell level. The methods described herein are well-adapted to use in mass cytometry and flow cytometry, and can also find use in techniques including, but not limited to imaging mass cytometry, multiplexed ion beam imaging (MIBI), and multiplexed ion beam imaging by time of flight (MIBI-TOF). While applicable to these and other single cell approaches, in an illustrative embodiment, described herein is a method of detecting a target molecule in mass cytometry, the method comprising: a) contacting a target-binding ligand molecule comprising a conjugated oligonucleotide primer with a cell or tissue sample under conditions permitting binding of the target-binding ligand molecule to its target in the cell or tissue sample; b) extending the oligonucleotide primer to comprise a concatemer of sequence comprised by the oligonucleotide primer; c) contacting the extended oligonucleotide primer of step (b) with a labeled probe nucleic acid comprising sequence complementary to sequence repeated in the concatemer, such that a plurality of labeled probe nucleic acid molecules hybridizes to a plurality of sequence repeats in the concatemer; d) cross-linking the hybridized probe nucleic acid molecules to the extended oligonucleotide primer; and e) detecting labeled probe using mass cytometry.

In one embodiment of this and any other aspect described herein, the labeled probe nucleic acid comprises a metal ion label. In one embodiment, the metal is a stable isotope of a lanthanide metal. Examples include isotopes of lanthanum, cerium, praseodymium, neodymium, promethium, europium, gadolinium, terbium, dysprosium, holmium, erbium, thulium, ytterbium and lutetium. Other metals/metal ions of potential use as distinguishable labels in, e.g., mass cytometry or related approaches can include, for example, isotopes of cadmium, palladium, indium, platinum, bismuth, selenium, tellurium, gold, silver, tantalum, yttrium, ruthenium, rhodium, iodine, osmium and iridium, among others. The mass spectra of the lanthanide metals are readily distinguishable, making them well-suited for multiplex applications in mass cytometry.

In another embodiment of this and any other aspect described herein, the labeled probe comprises a fluorophore. In another embodiment, the labeled probe can comprise a fluorophore label and a metal ion label.

In another embodiment of this and any other aspect described herein, extending the oligonucleotide primer comprises thermal cycling with a single-stranded extender template and a template-dependent nucleic acid polymerase enzyme.

In another embodiment of this and any other aspect described herein, the polymerase enzyme is thermostable.

In another embodiment of this and any other aspect described herein, the single-stranded extender template comprises a concatemer of at least two head-to-tail copies of a sequence complementary to the oligonucleotide primer, and a 3′ chain terminator, such that the extender template is not extended by the polymerase.

In another embodiment of this and any other aspect described herein, extending step (b) comprises a plurality of cycles of extension, thermal strand separation, and cooling to permit single-stranded extender annealing and extension by the polymerase, whereby successive cycles add successive concatemeric repeats to the oligonucleotide primer.

In another embodiment of this and any other aspect described herein, the method of detecting a target molecule in mass cytometry is performed in multiplex with a plurality of different target-binding ligand molecules, orthogonal sets of single-stranded extender templates, and distinguishably labeled orthogonal probe nucleic acids.

In another embodiment of this and any other aspect described herein, the concatemer comprises a branched concatemer, formed on or associated with the extended oligonucleotide conjugated to the target-binding ligand. Various branching approaches are described herein below, and can further increase the number of sites for hybridization of a nucleic acid probe on the target-binding ligand.

In another embodiment of this and any other aspect described herein, the method of detecting a target molecule in mass cytometry further comprises the step, before detection step (e), of imaging fluorophore associated with the cell or tissue sample.

In another embodiment of this and any other aspect described herein, cross-linking the hybridized probe nucleic acid molecules to the extended oligonucleotide primer comprises photo cross-linking. In another embodiment, the probe nucleic acid molecules comprise a photo cross-linking agent. In another embodiment, the photo cross-linking agent comprises 3-cyanovinylcarbazole phosphoramidite (CNVK), and cross-linking comprises UV irradiation.

In another embodiment of this and any other aspect described herein, the target-binding ligand comprises an antibody or antigen-binding fragment thereof.

In another aspect, described herein is a method of fluorescence-activated cell sorting, the method comprising: a) providing a sample comprising fixed cells; b) contacting a target-binding ligand molecule comprising a conjugated oligonucleotide primer with cells in the sample under conditions permitting binding of the target-binding ligand molecule to its target in or on the cells; c) extending the oligonucleotide primer to comprise a concatemer of sequence comprised by the oligonucleotide primer; d) contacting the extended oligonucleotide primer of step (c) with a fluorescently labeled probe nucleic acid comprising sequence complementary to sequence repeated in the concatemer, such that a plurality of labeled probe nucleic acid molecules hybridizes to a plurality of sequence repeats in the concatemer; e) cross-linking the hybridized probe nucleic acid molecules to the extended oligonucleotide primer; and f) sorting the cells via fluorescence-activated cell sorting.

In one embodiment of this and any other aspect described herein, the labeled probe further comprises a metal ion.

In another embodiment of this and any other aspect described herein, extending the oligonucleotide primer comprises thermal cycling with a single-stranded extender template and a template-dependent nucleic acid polymerase enzyme.

In another embodiment of this and any other aspect described herein, the polymerase enzyme is thermostable.

In another embodiment of this and any other aspect described herein, the single-stranded extender template comprises a concatemer of at least two head-to-tail copies of a sequence complementary to the oligonucleotide primer, and a 3′ chain terminator, such that the extender template is not extended by the polymerase.

In another embodiment of this and any other aspect described herein, extending step (c) comprises a plurality of cycles of extension, thermal strand separation, and cooling to permit single-stranded extender annealing and extension by the polymerase, whereby successive cycles add successive concatemeric repeats to the oligonucleotide primer.

In another embodiment of this and any other aspect described herein, the method is performed in multiplex with a plurality of different target-binding ligand molecules, orthogonal sets of single-stranded extender templates, and distinguishably labeled orthogonal probe nucleic acids.

In another embodiment of this and any other aspect described herein, the concatemer comprises a branched concatemer, formed on a target-binding ligand via thermal cycling.

In another embodiment of this and any other aspect described herein, the method further comprises the step, after sorting step (f), of detecting metal ion-labeled probe via mass cytometry.

In another embodiment of this and any other aspect described herein, cross-linking the hybridized probe nucleic acid molecules to the extended oligonucleotide primer comprises photo cross-linking. In another embodiment, the probe nucleic acid molecules comprise a photo cross-linking moiety. In another embodiment, the photo cross-linking moiety comprises 3-cyanovinylcarbazole phosphoramidite (CNVK), and cross-linking comprises UV irradiation. Other cross-linking agents include, but are not limited to psoralens (UV-activated), bromo-modification-based UV cross linking (e.g., 5-bromo deoxyuridine-mediated cross-linking), carmustine cross-linking, and click chemistry-mediated cross-linking.

In another embodiment of this and any other aspect described herein, the target-binding ligand comprises an antibody or antigen-binding fragment thereof. In another embodiment, the target-binding ligand comprises a DNA or RNA molecule, and can include, e.g., modified bases or linkages.

In another aspect, described herein is a composition comprising a target-binding ligand and a concatemeric oligonucleotide conjugated to the target-binding ligand.

In another embodiment of this and any other aspect described herein, the concatemeric oligonucleotide further comprises one or more branched concatemeric oligonucleotides.

In another embodiment of this and any other aspect described herein, the composition further comprises a plurality of nucleic acid probe molecules hybridized to concatemeric repeats of the concatemeric oligonucleotide.

In another embodiment of this and any other aspect described herein, the nucleic acid probe molecules comprise a cross-linking moiety. In one embodiment, the cross-linking moiety is a photo cross-linking moiety. Om another embodiment, the cross-linking moiety comprises 3-cyanovinylcarbazole phosphoramidite (CNVK).

In another embodiment of this and any other aspect described herein, the nucleic acid probe molecules are cross-linked to the concatemeric repeats.

In another embodiment of this and any other aspect described herein, the nucleic acid probe molecules comprise a fluorescent label or a metal ion label.

In another embodiment of this and any other aspect described herein, the nucleic acid probe molecules comprise a fluorescent label and a metal ion label.

In another embodiment of this and any other aspect described herein, the target-binding ligand comprises an antibody or antigen-binding fragment thereof.

In another aspect, described herein is a cell or tissue sample comprising a composition as described herein, wherein the target-binding ligand is bound to a target molecule in or on the cell or tissue sample.

In another aspect, described herein is a kit for performing one or more of the methods as described herein, the kit comprising: a) a target-binding ligand conjugated to an oligonucleotide primer; or a set of target-binding ligands conjugated to orthogonal oligonucleotide primers; b) a single-stranded extender template or a set of orthogonal single-stranded extender templates; c) a labeled probe nucleic acid or a set of orthogonal labeled probe nucleic acids, wherein the labeled probe nucleic acid or set of orthogonal labeled probe nucleic acids comprises a cross-linking moiety.

In one embodiment of this and any other aspect described herein, each single-stranded extender template comprises a concatemer of at least two head-to-tail copies of a sequence complementary to the oligonucleotide primer, and a 3′ chain terminator, such that the extender template is not extended by a polymerase.

In another embodiment of this and any other aspect described herein, the kit further comprises a template-dependent nucleic acid polymerase enzyme. In one embodiment, the polymerase enzyme is thermostable.

In another embodiment of this and any other aspect described herein, the target-binding ligand comprises an antibody or antigen-binding fragment thereof.

In another embodiment of this and any other aspect described herein, the orthogonal nucleotide primers, single stranded extender templates and nucleic acid probes are optimized to avoid primer/extender cross-talk, primer-dimer formation, and off-target hybridization.

In another embodiment of this and any other aspect described herein, the single-stranded extender template or the set of single-stranded extender templates are suitable for performing a concatemer-generating method.

In another embodiment of this and any other aspect described herein, the kit further comprises packaging materials for the various components and instructions for use.

In another embodiment of this and any other aspect described herein, the kit further comprises one or more target-binding ligand molecules or reagents for conjugating a first oligonucleotide to a target-binding ligand.

In another embodiment of this and any other aspect described herein, the kit further comprises a thermostable polymerase, nucleotides, reaction buffer components, and reagents for labeling a nucleic acid probe molecule.

In another embodiment of this and any other aspect described herein, the probe molecule can be complementary to a concatemer repeat element.

In another embodiment of this and any other aspect described herein, the cross-linking moiety comprises a photo cross-linking moiety. In one embodiment, the cross-linking moiety comprises 3-cyanovinylcarbazole phosphoramidite (CNVK).

In another embodiment of this and any other aspect described herein, the labeled probe nucleic acid or set of orthogonal labeled probe nucleic acids comprises a fluorescent label or a metal ion label.

In another embodiment of this and any other aspect described herein, the labeled nucleic acid probe molecules comprise a fluorescent label and a metal ion label.

### Definitions

As used herein, the term “target-binding ligand” refers to a molecule or moiety that specifically binds a given target molecule. Target-binding ligands can include, for example, peptides, polypeptides, nucleic acids, aptamers, a receptor and/or its cognate ligand, members of an affinity binding pair (including, but not limited to biotin/streptavidin), and small molecule agents that specifically bind a target molecule as that term is defined herein. Antibodies and antigen-binding fragments or constructs thereof represent one class of target-binding ligands useful in the methods, compositions and kits described herein. Nucleic acids comprising sequence complementary to a given target DNA or RNA molecule (including, but not limited to an mRNA molecule) represent another class of target-binding ligands that can be useful in the methods, compositions and kits described herein.

As used herein, the term “specific binding” refers to a physical interaction between two molecules, compounds, cells and/or particles wherein the first entity binds to the second, target entity with greater specificity and affinity than it binds to a third entity which is a non-target. A reagent specific for a given target is one that exhibits specific binding for that target under the conditions of the assay being utilized. The specificity of an antibody or antibody fragment thereof can be determined based on affinity and/or avidity. The affinity, represented by the equilibrium constant for the dissociation (KD) of an antigen with an antigen-binding protein, is a measure of the binding strength between an antigenic determinant and an antigen-binding site on the antigen-binding protein, such as an antibody or antigen-binding fragment thereof: the less the value of the KD, the stronger the binding strength between an antigenic determinant and the antigen-binding molecule. Alternatively, the affinity can also be expressed as the affinity constant (KA), which is 1/K_{D}). Accordingly, an antibody or antigen-binding fragment thereof as described herein is said to be “specific for” or to “specifically bind” or “selectively bind” a first target or antigen compared to a second target or antigen when it binds to the first antigen with an affinity (as described above, and suitably expressed, for example as a K_{D} value) that is at least 1000 times, 10000 times or more better than the affinity with which said amino acid sequence or polypeptide binds to another given polypeptide. Generally, a molecule that “specifically binds,” “selectively binds” or “is specific for” a given target will bind with a K_{D} of 10⁻⁵ M (10000 nM) or less, e.g., 10⁻⁶ M, 10⁻⁷ M, 10⁻⁸ M, 10⁻⁹ M, 10⁻¹⁰ M, 10⁻¹¹ M, 10⁻¹² M, or less. Specific binding can be influenced by, for example, the affinity and avidity of the polypeptide agent and the concentration of polypeptide agent. The person of ordinary skill in the art can determine appropriate conditions under which polypeptide agents as described herein selectively bind the target using any suitable methods, such as titration of a polypeptide agent in a suitable cell binding assay.

It should be understood in this context that where antibodies or antigen-binding fragments thereof are concerned, the specific binding is mediated by the CDRs of the antibody polypeptide, as opposed to any other portion of the antibody polypeptide. Antibody dissociation constants and affinities can be determined, for example, by a surface plasmon resonance based assay (such as the BIAcore assay described in PCT Application Publication No. WO2005/012359); Forte Bio Octet™ analysis, enzyme-linked immunosorbent assay (ELISA); and competition assays (e.g., RIA's), for example.

As used herein, the term “conjugated” refers to the linkage of, for example, an oligonucleotide to a target-binding ligand in a manner that is stable through steps of thermal cycling to generate concatemers or ordered nucleic acid extension products as described herein. Conjugates can include covalent linkages. In various embodiments, conjugates can include a linker molecule between the target-binding ligand and the conjugated oligonucleotide.

As used herein the term “concatemer” refers to a nucleic acid molecule comprising two or more repeats of a given sequence in a head-to-tail, 5′ to 3′ orientation. Concatemers can include two or more, three or more, four or more, five or more, six or more, seven or more, eight or more, nine or more, ten or more, 15 or more, 20 or more, 25 or more, 30 or more, 35 or more, 40 or more, 45 or more, or 50 or more repeats of the given sequence. The number of repeats for concatemers generated as described herein is determined by the number of cycles of strand separation and primer extension employed. Repeat unit lengths of concatemers as described herein can determine the degree of multiplexing achievable for orthogonal sets of repeats of a given length. The repeat units in a concatemer as described herein can be, for example, 8 or more, 9 or more, 10 or more, 11 or more, 12 or more, 13 or more, 14 or more, 15 or more, 16 or more, 17 or more, 18 or more, 19 or more, 20 or more, 25 or more, 30 or more, 35 or more, 40 or more, 45 or more, or 50 or more nucleotides in length.

As used herein, the term “conditions permitting hybridization and extension” of a given molecule, e.g., an oligonucleotide molecule, refers to conditions of temperature, salt, buffer and other reaction components sufficient for a template-directed polymerase-mediated primer extension reaction. Exact conditions for extension by a given polymerase vary with the enzyme chosen, but are known in the art and/or can be determined without undue experimentation. Conditions for hybridization, e.g., of a primer as described herein to a single-stranded extender template as described herein, will generally be those salt, buffer and other reaction component conditions appropriate for the chosen polymerase enzyme, with the annealing or hybridization temperature established by one of skill in the art primarily on the basis of the length of the concatemer repeat unit and degree of multiplexing in a given reaction. Thus, where performed in uniplex, with one given molecule being labeled with a single concatemeric sequence in the reaction, an annealing or hybridization temperature can be determined based on the Tm of the specific concatemer repeat sequence—generally the annealing temperature in a cycling reaction is about 5° C. below the Tₘ for the hybridization of the repeat sequence to its complement under the salt and reaction component concentrations optimal for the enzyme of choice. Where performed in multiplex, the length of the repeat unit takes on added importance, as the Tm for various repeat sequences will vary. Primer/single-stranded extended template design can take sequence variation into account to design primers and single-stranded extender templates that have Tₘ values that are relatively close to each other, generally on the order of within 5-7° C. for all members of a set of primer/extender template sequences. Under these circumstances, a single annealing or hybridization temperature in a cycling reaction can permit efficient hybridization and extension of members of the set in multiplex. In various embodiments of a method including a step of contacting a nucleic acid molecule with a single-stranded extender template under conditions permitting hybridization and extension of the nucleic acid molecule, the reaction can be incubated at an annealing temperature for a period of time (generally a matter of seconds to minutes) before raising the temperature to an optimum extension temperature for the polymerase enzyme. It should be understood that contacting a nucleic acid molecule with a single-stranded extender template “under conditions permitting hybridization and extension” of the nucleic acid molecule can include such an annealing/extending temperature shift. In some embodiments, annealing or hybridization occurs efficiently at a temperature at which the polymerase enzyme will be sufficiently active as not to require such a temperature shift; whether or not such a shift is needed in a given circumstance will be apparent to the one of ordinary skill in the art depending upon the enzyme used, and can also be determined empirically without undue experimentation.

### BRIEF DESCRIPTION OF THE DRAWINGS

FIG. 1A-1B. FIG. 1A is a schematic of epitope detection with conventional mass cytometry antibodies. FIG. 1B is a schematic of epitope detection with ACED (amplification by cyclic extension of DNA oligo) amplification method.

FIG. 2 shows CNvK-based photocrosslinking allow hybridized detection strands to stay intact during mass cytometry sample acquisition.

FIG. 3A-3C. FIG. 3A Transient transfection was used to generate an expression gradient of GFP in a population of cells that were subsequently targeted by an anti-GFP antibody conjugated with ACED oligos. Signal amplification by ACED (y-axis) through 1-500 thermal cycles was compared with a counterstaining using anti-rat secondary antibodies to show the specificity of ACED. FIG. 3B examines data in FIG. 3A was divided into 10 equal-width bins according to their GFP expression level showed by the secondary antibody. Bin 1 indicates the untransfected cells as internal control. Bin 10 shows the cells with the highest GFP expression level. FIG. 3C shows bin medians and signal-to-noise ratio (i.e., medians of bin 1-9 compared to medians in bin 1) across the 1-500 thermal cycles are demonstrated in the left two plots, indicating a 13-fold amplification strength and a six-fold signal-to-noise ratio enhancement. Fold change for each bin through the 1-500 cycles are plotted as a bar graph on the right.

FIG. 4 depicts signal amplification by one or two rounds of branching were compared to that from a linear amplification without branching. A 17-fold amplification for each branching round could be observed.

FIG. 5 examines how ACED showed high orthogonality by pairwise analysis of six extenders and metal strands.

FIG. 6 analyzes ACED mass cytometry was performed on Py2T cells that were undergoing EMT. Samples were analyzed without treatment or with 3, 5, or 7 days of TGFβ treatment. 30 EMT markers, including E-cadherin, vimentin, Zeb1, Snail/Slug, Smad2/3, and Smad4 were simultaneously analyzed to reveal the EMT states.

FIG. 7A-7C. Schemes for thermal controllable DNA extension. FIG. 7A shows thermal cycling extension (TCE) can be used to generate concatemers with repeated sequences. FIG. 7B shows TCE to achieve multiplexed concatemer generation. FIG. 7C shows TCE can also be used for primer exchange reactions.

FIG. 8A-8B shows enhanced signal amplification by branching ACE amplification. FIG. 8A examines after the linear extension (Step 1), the primary detection strand can be applied directly for mass cytometry analysis (Step 2). When branching is required to further amplify signals, the number of detection strand binding sites on each antibody can be expanded by applying the CNvK-containing primary branching strand (Step 3), followed by UV crosslinking and thermal cyclic primer extension (Step 4). The secondary detection strand can then be applied before mass cytometry analysis (Step 5). Based on the same principle, secondary branching (Step 6-8) can be implemented to ultimately achieve over 500-fold signal enhancement, compared to the unamplified signal. FIG. 8B examines GFP ion counts generated with ACE after one or two rounds of branching were compared to that from a linear amplification without branching in a ERK2-GFP transient expression experiment using ultra-low amount of the GFP antibody (10 ng/ml). ERK2-GFP-expressing cells were stained with oligo-conjugated rat anti-GFP antibodies for linear or branching ACE amplifications, followed by ¹⁴¹Pr-labeled detection strand hybridization. Conventionally labeled ¹⁷²Yb anti-ERK2 antibodies were used to detect the ERK2-GFP fusion proteins in the same cells, which were later analyzed by mass cytometry. We observed that primary branching amplification with a 50 thermal-cycle branching strand extension step over 1 hour generated a further 9-fold signal enhancement, compared to linear amplification. In addition, a secondary branching approach could also be applied, resulting in an additional 5-fold signal increase, allowing an initial unamplified signal to be increased more than 500-fold.

FIG. 9A-9B depicts how ACE showed high orthogonality with an overall 1.02% crosstalk signal in a 33-plex multiplexity assessment. FIG. 9A shows GFP-expressing HEK293T cells were individually stained with anti-GFP ACE antibodies conjugated to 33 initiator sequences before they were barcoded, pooled, and processed through the ACE protocol in the same tube before they were analyzed on a mass cytometer. FIG. 9B shows data were then de-barcoded to allow pairwise analysis of potential ACE crosstalk. Ion counts generated by a detection strand in all conditions (each column) were normalized to 0-1 before the ratio between any unmatched initiator-detector pair (e.g., Ab-initiator 1—Detector 2′—¹⁴²Nd) and the true signal (e.g., Ab-initiator 1—Detector 1′—¹⁴¹Pr) was calculated. On average, ACE has 1.02% of crosstalk signal. Four pairs of probes were detected with crosstalk degrees over 10% (Initiator 2—Detector 3′, Initiator 4—Detector 5′, Initiator 7—Detector 8′, Initiator 20—Detector 21′).

FIG. 10 shows GFP+ and GFP− E. coli cells stained using an DNA barcode-conjugated anti-GFP antibody. With two rounds of branching, protein abundances in microbes could be measured using mass cytometry.

FIG. 11A depicts in primary human CD4 T cells, p-ERK signals were only slightly above the mass cytometer detection limit using conventionally conjugated antibodies. With ACE, p-ERK signals were drastically increased that showed expected trend during a TCR stimulation time course experiment. FIG. 11B analyzes primary human T cells harvested over a 1-hour TCR stimulation time course, ACE revealed differential signaling responses on key TCR signaling mediators, including p-CD3ζ, p-CD28, p-ZAP70/SYK, p-LAT, p-SLP76,p-PLCγ1, p-BTK/ITK, p-MEK1/2, p-ERK1/2, p-p90RSK and p-S6.

FIG. 12A shows high-abundance proteins may be captured to generate detectable signals in the IMC analysis. Due to this limitation, recent IMC-based human breast cancer studies were designed focusing on cell surface epitopes, cytokeratin proteins, and a few cellular functional indicators. FIG. 12B depicts that by applying ACE to amplify IMC signal, a wide selection of low-abundance targets can be detectable on an IMC machine. FIG. 12C depicts by applying two rounds of branching, ACE is expected to increase the averaged metal ion counts by nearly 4000-fold. The laser ablation crater size can, therefore, be reduced to a sub-diffraction-limit level of 100 nm in diameter, compared to the currently used 1 μm² laser beam.

FIG. 13A shows human kidney sections with 20 ACE oligo conjugated antibodies simultaneously. After post-staining fixation, these markers were amplified with linear or branching ACE before analyzing the slides on IMC. FIG. 13B shows 18 of the ACE amplified kidney markers were shown in three overlaid images where each marker was indicated by a specific color. Locations of these markers in the kidney tissues were as expected. FIG. 13C shows single-cell segmentation was performed and segmented cells were embedded onto a two-dimensional UMAP plot. FIG. 13D shows cell types detected showed unique protein expression profile that can be only studied with multiplex spatial proteomic analysis powered by ACE.

### DETAILED DESCRIPTION

The technology described herein relates to improvements in the ability to detect target molecules at a single cell level at high sensitivity and optionally in multiplex. Certain embodiments of the technology described herein involve the generation of a concatemeric nucleic acid on a target-binding molecule, which can be in situ, e.g., bound to a target molecule in or on a cell. By providing binding sites or “landing pads” for multiple copies of a probe complementary to a concatemeric repeat element, the technology labels the target-binding molecule in a manner permitting detection of even low-abundance target molecules, which, in some embodiments, can include even single molecule sensitivity. The methods are applicable in a range of different scenarios, but find particular application in, for example, mass cytometry and flow cytometry, among others.

The following describes various considerations involved in practicing the technology disclosed herein.

### Preparation of Cell Samples:

Mass cytometry, flow cytometry and similar methods are best adapted to the analysis of single-cell suspensions. Non-limiting examples can include suspension cultured cells, adherent cultured cells placed in single-cell suspension (e.g., by trypsin digest), cells in a blood sample, and cells dissociated from a tissue (healthy or diseased, including but not limited to tumor tissue), e.g., by physical and/or enzymatic methods, among others. Cell samples can be fixed to avoid deterioration over subsequent staining and detection steps. The sample can be fixed as soon after collection as possible. There are many different types of fixatives known in the art. Exemplary fixatives include, but are not limited to, paraformaldehyde (PFA) at various concentrations (commonly between 1% and 5%, e.g., 1%, 1.2%, 1.4%, 1.6%, 1.8%, 2.0%, 2.2%, 2,4%, 2.6%, 2.8%, 3.0%, 3.2%, 3.4%, 3.6%, 3.8%, 4%, 4.2%, 4.4%, 4.6%, 4.8% or 5.0%), formaldehyde at various concentrations (e.g., 2%, 2.5%, 3%, 3.5%, 4%, 4.5% or 5% formaldehyde), 10% neutral buffered formalin, Bouin's solution, methanol, acetone, glutaraldehyde, etc. The cell or tissue sample can be treated or processed so as to minimize nucleic acid degradation, where, for example, RNA is the target molecule. One of ordinary skill in the art will be able to determine the fixative best suited for the cell or tissue sample and technique to be performed. In some embodiments, the fixative that can be used is 1.6% paraformaldehyde.

In further detail, for a cultured cell line, paraformaldehyde (PFA, from Electron Microscopy Sciences) can be added to the cell suspension to a final percentage of 1.6%, and cells incubated at room temperature for 10 min. Crosslinked cells are washed twice with cell staining media (CSM, PBS with 0.5% BSA, 0.02% NaN3) and after centrifugation, ice-cold methanol is used to resuspend the cells, followed by a 10-min permeabilization on ice or for long-term storage at −80° C.

For a tumor cell sample, following surgical resection, fresh tissue samples can be immediately transferred to pre-cooled MACS Tissue Storage Solution (Miltenyi Biotec) and shipped at 4° C. Tissue processing is best completed within 24 hours of collection. For dissociation, the tissue is minced using surgical scalpels and further disintegrated using the Tumor Dissociation Kit, human (Miltenyi Biotech) and the gentleMACS Dissociator (Miltenyi Biotech) according to manufacturer's instructions. The resulting single-cell suspension is filtered sequentially through sterile 70-μm and 40-μm cell strainers. The cell suspension is stained for viability with 25 μM cisplatin (Enzo Life Sciences) in a 1-min pulse before quenching with 10% FBS. Cells are then fixed with 1.6% paraformaldehyde (PFA, Electron Microscopy Sciences) for 10 min at room temperature and stored at −80° C.

For human monocytes, the monocytes can be isolated by histopaque (Sigma Aldrich) density gradient centrifugation followed by a MACS purification using the pan monocyte isolation kit (Miltenyi Biotech) according to manufacturer's instructions. Monocytes are differentiated into immature macrophages by culture for 5 days in 75-cm² tissue culture dishes in presence of 30 ng/ml M-CSF (PeproTech). Cells are subsequently polarized for 24 hr. For mass cytometry analysis, cells are stained for viability with 10 μM cisplatin (Enzo Life Sciences) in a 1 min pulse before quenching with 10% FBS as previously described (Fienberg et al., 2012). Cells are then fixed with 1.6% paraformaldehyde (Electron Microscopy Sciences) for 10 min at room temperature and stored at −80° C.

### Target-Binding Ligands:

In some embodiments of any of the aspects, an oligonucleotide primer strand is attached to a target-binding ligand molecule.

As used herein, a “target-binding ligand” is a molecule or moiety that binds, e.g., specifically binds, to a target molecule of interest. The target-binding ligand can be a synthetic or natural molecule. A target-binding ligand can be a biomolecule, such as a polypeptide or a polynucleotide. In some embodiments, a target-binding ligand is a polypeptide. In some embodiments, a target-binding ligand is a protein or fragment thereof. Non-limiting examples of target-binding ligands include peptides, polypeptides, antibodies and antibody derivatives, members of an affinity-binding pair (e.g., avidin/streptavidin), oligonucleotides, aptamers and receptors.

In some embodiments of any of the aspects, the target-binding ligand binds to i.e., the target molecule is, a molecule selected from the non-limiting group of lipids, sugars, oligo- or poly-saccharides, amino acids, peptides or polypeptides, nucleosides, nucleotides, oligo- or poly-nucleotides, hormones, vitamins, small molecules, miRNAs, metabolites, and any combinations thereof.

In some embodiments of any of the aspects, the target-binding ligand binds a molecule that is DNA or RNA barcoded.

In some embodiments of any of the aspects, the target binding molecule is an antibody. The term “antibody” refers to a protein that includes at least one immunoglobulin variable domain or immunoglobulin variable domain sequence that binds a target molecule. For example, an antibody can include an immunoglobulin heavy (H) chain variable region (abbreviated herein as V_{H}), and an immunoglobulin light (L) chain variable region (abbreviated herein as V_{L}). In another example, an antibody includes two heavy (H) chain variable regions and two light (L) chain variable regions. An antibody can include the structural features of IgA, IgG, IgE, IgD, IgM (as well as subtypes thereof). The term antibody as used herein includes any of a number of different constructs using one or more antigen-binding domains or fragments of an antibody to mediate binding to a target molecule. Thus, in addition to a complete IgA, IgG, IgE, IgD or IgM antibody, an antibody includes, but is not limited to antigen-binding fragments of antibodies (e.g., single chain antibodies, Fab and sFab fragments, F(ab')2, Fd fragments, Fv fragments, scFv, domain antibodies (dAb) (de Wildt et al., Eur J Immunol. 1996; 26(3):629-39) and nanobodies. An affibody, which uses a non-antibody scaffold to support a diverse target-binding domain, can also be used as a target-binding ligand in the methods, compositions and kits described herein.

As used herein, an “antigen-binding fragment” refers that portion of an antibody that is necessary and sufficient for binding to a given antigen. At a minimum, an antigen binding fragment of a conventional antibody will comprise six complementarity determining regions (CDRs) derived from the heavy and light chain polypeptides of an antibody arranged on a scaffold that permits them to selectively bind the antigen. A commonly used antigen-binding fragment includes the V_{H} and V_{L} domains of an antibody, which can be joined either via part of the constant domains of the heavy and light chains of an antibody, or, alternatively, by a linker, such as a peptide linker. Non-conventional antibodies, such as camelid and short antibodies have only heavy chain sequences, denoted, for example V_{HH}. These can be used in a manner analogous to V_{H}/V_{L}-containing antigen-binding fragments. Non-limiting examples of antibody fragments encompassed by the term antigen-binding fragment include: (i) a Fab fragment, having V_{L}, C_{L}, V_{H} and C_{H}1 domains; (ii) a Fab' fragment, which is a Fab fragment having one or more cysteine residues at the C-terminus of the C_{H}1 domain; (iii) an Fd fragment having V_{H} and C_{H}1 domains; (iv) a Fd' fragment having V_{H} and C_{H}1 domains and one or more cysteine residues at the C-terminus of the CHI domain; (v) an Fv fragment having the V_{L} and V_{H} domains of a single arm of an antibody; (vi) a dAb fragment (Ward et al., Nature 341, 544-546 (1989)) which consists of a V_{H} domain; (vii) F(ab')₂ fragments, a bivalent fragment including two Fab' fragments linked by a disulphide bridge at the hinge region; (viii) single chain antibody molecules (e.g., single chain Fv; scFv) (Bird et al., Science 242:423-426 (1988); and Huston et al., PNAS (USA) 85:5879-5883 (1988)); (ix) “diabodies” with two antigen binding sites, comprising a heavy chain variable domain (V_{H}) connected to a light chain variable domain (V_{L}) in the same polypeptide chain (see, e.g., EP 404,097; WO 93/11161; and Hollinger et al., Proc. Natl. Acad. Sci. USA, 90:6444-6448(1993)); and “linear antibodies” comprising a pair of tandem Fd segments (V_{H}-C_{H}1-V_{H}-C_{H}1) which, together with complementary light chain polypeptides, form a pair of antigen binding regions (Zapata et al. Protein Eng. 8(10): 1057-1062 (1995); and U.S. Pat. No. 5,641,870). The molecules of Fv, scFv or diabody can be stabilized by incorporating disulfide bridges linking the V_{H} and V_{L} domains. Minibodies comprising a scFv fragment linked to a CH3 domain can also be obtained. Other examples of binding fragments are Fab', which differs from Fab fragments by the addition of some residues at the carboxyl terminus of the CH1 domain of the heavy chain, including one or more cysteines of the hinge region of the antibody, and Fab'-SH, which is a Fab' fragment in which the cysteine residue(s) of the constant domains carries a free thiol group.

### Oligonucleotides:

Target-binding ligands as described herein include a single-stranded oligonucleotide primer that permits concatemer generation according to the methods described. Single-stranded oligonucleotide primers can be attached to target-binding ligands by any of various approaches known in the art. Note that while the single-stranded oligonucleotide can be DNA, RNA, or modified forms of either, for ease of reference and proof of principle, DNA oligonucleotides are used in the description and Examples that follow.

Oligonucleotides and oligonucleotide synthesis are well known in the art, and a number of different commercial sources provide custom oligonucleotide synthesis, such that an oligonucleotide with essentially any sequence can be readily obtained. Oligonucleotides are commercially available for custom design and use from Eurofins Genomics (Louisville, KY), ThermoFisher (Waltham, MA), Integrated DNA Technologies (Coralville, Iowa), TriLink Biotechologies (San Diego, CA) and the like. Furthermore, it is known in the art how to generate oligonucleotides that include modified nucleosides and/or modified linkages, and oligonucleotides including any of a number of such modifications are also commercially available. To be clear, an oligonucleotide “primer” is an oligonucleotide that can be extended by a template-dependent nucleic acid polymerase when hybridized (e.g., via hydrogen-bonded base pairing) to a template nucleic acid molecule. A single-stranded extender template oligonucleotide as described herein has a 3′ modification or moiety (also referred to as a “stopper” moiety) that blocks or precludes template-dependent extension. Such a modification can also be referred to as a chain terminator, and includes, for example, a dideoxy nucleoside, 3′-CPR II CPG, 3′-phosphate CPG, 5′-Ome-dT-CE Phosphoramidite, 5′-amino-dT-CE Phosphoramidite, 2′3′-ddA-CE Phosphoramidite, 2′3′-ddC-CE Phosphoramidite, 2′3-ddG-CE Phosphoramidite, 2′3′-ddT-CE Phosphoramidite, 3′-dA-CPG, 3′-ddC-CPG, 3′-dC-CPG, 3′-dG-CPG, 3′-dT-CPG, 3′Spacer C3 CPG and the like. In some embodiments, oligonucleotides can also include, for example, nucleosides modified to include cross-linking moieties. The inclusion of a cross linking moiety, including but not limited to a photo cross-linking moiety, can permit, for example, the cross-linking of an oligonucleotide to a target sequence such that the oligonucleotide does not dissociate in subsequent processes, such as thermal cycling to generate concatemers. Thus, in some embodiments of any one of the aspects, an oligonucleotide strand comprises a photo-cross linking moiety, for example, a photo-cross linking moiety selected from the group consisting of 3-Cyanovinylcarbazole (CNVK) nucleotide; 5-bromo deoxycytosine; 5-iodo deoxycytosine; 5-bromo deoxyuridine (Bromo dU); 5-iodo deoxyuridine; and nucleotides comprising an aryl azide (AB-dUMP), benzophenone (BP-dUMP), perfluorinated aryl azide (FAB-dUMP) or diazirine (DB-dUMP), psoralen, 4-thio-dT (S4dT), and the like.

Oligonucleotides useful in the methods, compositions and kits described herein will generally be at least 9 nucleotides in length, but can vary from 8 to 100 or more nucleotides in length. Thus, an oligonucleotide useful in the methods, compositions or kits described herein can be, for example, between 9-100 nucleotides in length, between 9-95 nucleotides in length, between 9-90 nucleotides in length, between 9-85 nucleotides in length, between 9-80 nucleotides in length, between 9-75 nucleotides in length, between 9-70 nucleotides in length, between 9-65 nucleotides in length, between 9-60 nucleotides in length, between 9-55 nucleotides in length, between 9-50 nucleotides in length, between 9-45 nucleotides in length, between 9-40 nucleotides in length, between 9-35 nucleotides in length, between 9-30 nucleotides in length, between 9-25 nucleotides in length, between 9-20 nucleotides in length, between 9-15 nucleotides in length, between 10-100 nucleotides in length, between 10-90 nucleotides in length, between 10-80 nucleotides in length, between 10-70 nucleotides in length, between 10-60 nucleotides in length, between 10-50 nucleotides in length, between 10-40 nucleotides in length, between 10-30 nucleotides in length, between 10-20 nucleotides in length, between 11-100 nucleotides in length, between 11-90 nucleotides in length, between 11-80 nucleotides in length, between 11-70 nucleotides in length, between 11-60 nucleotides in length, between 11-50 nucleotides in length, between 11-40 nucleotides in length, between 11-30 nucleotides in length, between 11-20 nucleotides in length, between 12-100 nucleotides in length, between 12-90 nucleotides in length, between 12-80 nucleotides in length, between 12-70 nucleotides in length, between 12-60 nucleotides in length, between 12-50 nucleotides in length, between 12-40 nucleotides in length, between 12-30 nucleotides in length, between 12-20 nucleotides in length, between 13-100nucleotides in length, between 13-90 nucleotides in length, between 13-80 nucleotides in length, between 13-80 nucleotides in length, between 13-70 nucleotides in length, between 13-60 nucleotides in length, between 13-50 nucleotides in length, between 13-40 nucleotides in length, between 13-30 nucleotides in length, between 13-20 nucleotides in length, between 14-100 nucleotides in length, between 14-90 nucleotides in length, between 14-80nucleotides in length, between 14-70 nucleotides in length, between 14-60 nucleotides in length, between 14-50 nucleotides in length, between 14-40 nucleotides in length, between 14-30 nucleotides in length, between 14-20 nucleotides in length, between 15-100nucleotides in length, between 15-90 nucleotides in length, between 15-80 nucleotides in length, between 15-70 nucleotides in length, between 15-60 nucleotides in length, between 15-50 nucleotides in length, between 15-40 nucleotides in length, between 15-30 nucleotides in length, or between 15-20 nucleotides in length.

Methods for the conjugation of oligonucleotides to antibodies are known in the art. See, e.g., Dugal-Tessier et al., J. Clin. Med. 10:838 (2021) for a review discussing various methods. Kits are available for the conjugation of oligonucleotides to antibodies; see, e.g., Abcam Oligonucleotide Conjugation Kit (ab218260), which permits conjugation to the 5′ or 3′ end of oligos from 10 to 120 nucleotide long. Approaches appropriate for the conjugation of an oligonucleotide to a non-antibody target-binding ligand will vary depending up on the ligand, but are known to those of ordinary skill in the art. Single-stranded oligonucleotide primers are conjugated with target-binding ligand in a manner that permits template-directed nucleic acid polymerase extension from the 3′ end of the primer. While various approaches for primer conjugation can be used, it is therefore important that the method used keep the 3′ nucleotide open for extension. Examples include conjugation via linking moiety attached at or near the 5′ end of the oligonucleotide primer.

Included among oligonucleotides as described herein are nucleic acid probe molecules. In general, a nucleic acid probe molecule is an oligonucleotide that includes a label that is directly or indirectly detectable to provide a signal. Non-limiting examples of a label can include a fluorescent label or a metal, e.g. a lanthanide or other metal permitting detection in mass cytometry. Additional examples of detectable labels include, for example, isotopes, e.g., ³²P, ³⁵S, etc., quantum dots, organic dyes, polymer nanoparticles, metallic nanoparticles, and Raman dots among others. Methods of the preparation of labeled oligonucleotide probes bearing any of a number of different detectable moieties are known to those of skill in the art. Multiple copies of a nucleic acid probe that binds to a sequence repeated in a concatemer produced as described herein can bind to such a concatemer such that each molecule of target bound by a target-binding ligand as described herein has multiple label moieties associated with it, greatly amplifying the signal, such that the presence, location and/or amount of a given target can be determined to as much as single-molecule sensitivity.

A wide variety of fluorescent reporter dyes are known in the art. Typically, the fluorophore is an aromatic or heteroaromatic compound and can be a pyrene, anthracene, naphthalene, acridine, stilbene, indole, benzindole, oxazole, thiazole, benzothiazole, cyanine, carbocyanine, salicylate, anthranilate, coumarin, fluorescein, rhodamine or other like compound.

Exemplary fluorophores include, but are not limited to, 1,5 IAEDANS; 1,8-ANS; 4-Methylumbelliferone; 5-carboxy-2,7-dichlorofluorescein; 5-Carboxyfluorescein (5-FAM); 5-Carboxynapthofluorescein (pH 10); 5-Carboxytetramethylrhodamine (5-TAMRA); 5-FAM (5-Carboxyfluorescein); 5-Hydroxy Tryptamine (HAT); 5-ROX (carboxy-X-rhodamine); 5-TAMRA (5-Carboxytetramethylrhodamine); 6-Carboxyrhodamine 6G; 6-CR 6G; 6-JOE; 7-Amino-4-methylcoumarin; 7-Aminoactinomycin D (7-AAD); 7-Hydroxy-4-methylcoumarin; 9-Amino-6-chloro-2-methoxyacridine; ABQ; Acid Fuchsin; ACMA (9-Amino-6-chloro-2-methoxyacridine); Acridine Orange; Acridine Red; Acridine Yellow; Acriflavin; Acriflavin Feulgen SITSA; Aequorin (Photoprotein); Alexa Fluor 350™; Alexa Fluor 430™; Alexa Fluor 488™; Alexa Fluor 532™; Alexa Fluor 546™; Alexa Fluor 568™; Alexa Fluor 594™; Alexa Fluor 633™; Alexa Fluor 647™; Alexa Fluor 660™; Alexa Fluor 680™; Alizarin Complexon; Alizarin Red; Allophycocyanin (APC); AMC, AMCA-S; AMCA (Aminomethylcoumarin); AMCA-X; Aminoactinomycin D; Aminocoumarin; Anilin Blue; Anthrocyl stearate; APC-Cy7; APTS; Astrazon Brilliant Red 4G; Astrazon Orange R; Astrazon Red 6B; Astrazon Yellow 7 GLL; Atabrine; ATTO-TAG™ CBQCA; ATTO-TAG™ FQ; Auramine; Aurophosphine G; Aurophosphine; BAO 9 (Bisaminophenyloxadiazole); BCECF (high pH); BCECF (low pH); Berberine Sulphate; Beta Lactamase; BFP blue shifted GFP (Y66H); BG-647; Bimane; Bisbenzamide; Blancophor FFG; Blancophor SV; BOBO™-1; BOBO™-3; Bodipy 492/515; Bodipy 493/503; Bodipy 500/510; Bodipy 505/515; Bodipy 530/550; Bodipy 542/563; Bodipy 558/568; Bodipy 564/570; Bodipy 576/589; Bodipy 581/591; Bodipy 630/650-X; Bodipy 650/665-X; Bodipy 665/676; Bodipy Fl; Bodipy FL ATP; Bodipy Fl-Ceramide; Bodipy R6G SE; Bodipy TMR; Bodipy TMR-X conjugate; Bodipy TMR-X, SE; Bodipy TR; Bodipy TR ATP; Bodipy TR-X SE; BO-PRO™-1; BO-PRO™-3; Brilliant Sulphoflavin FF; Calcein; Calcein Blue; Calcium Crimson™; Calcium Green; Calcium Green-1 Ca2+Dye; Calcium Green-2 Ca2+; Calcium Green-5N Ca2+; Calcium Green-C18 Ca2+; Calcium Orange; Calcofluor White; Carboxy-X-rhodamine (5-ROX); Cascade Blue™; Cascade Yellow; Catecholamine; CFDA; CFP-Cyan Fluorescent Protein; Chlorophyll; Chromomycin A; Chromomycin A; CMFDA; Coelenterazine; Coelenterazine cp; Coelenterazine f; Coelenterazine fcp; Coelenterazine h; Coelenterazine hcp; Coelenterazine ip; Coelenterazine O; Coumarin Phalloidin; CPM Methylcoumarin; CTC; Cy2™; Cy3.18; Cy3.5TM; Cy3TM; Cy5.1 8; Cy5.5™; Cy5™; Cy7™; Cyan GFP; cyclic AMP Fluorosensor (FiCRhR); d2; Dabcyl; Dansyl; Dansyl Amine; Dansyl Cadaverine; Dansyl Chloride; Dansyl DHPE; Dansyl fluoride; DAPI; Dapoxyl; Dapoxyl 2; Dapoxyl 3; DCFDA; DCFH (Dichlorodihydrofluorescein Diacetate); DDAO; DHR (Dihydorhodamine 123); Di-4-ANEPPS; Di-8-ANEPPS (non-ratio); DiA (4-Di-16-ASP); DIDS; Dihydorhodamine 123 (DHR); DiO (DiOC18(3)); DiR; DIR (DiIC18(7)); Dopamine; DsRed; DTAF; DY-630-NHS; DY-635-NHS; EBFP; ECFP; EGFP; ELF 97; Eosin; Erythrosin; Erythrosin ITC; Ethidium homodimer-1 (EthD-1); Euchrysin; Europium (III) chloride; Europium; EYFP; Fast Blue; FDA; Feulgen (Pararosaniline); FITC; FL-645; Flazo Orange; Fluo-3; Fluo-4; Fluorescein Diacetate; Fluoro-Emerald; Fluoro-Gold (Hydroxystilbamidine); Fluor-Ruby; FluorX; FM 1-43™; FM 4-46; Fura Red™(high pH); Fura-2, high calcium; Fura-2, low calcium; Genacryl Brilliant Red B; Genacryl Brilliant Yellow 10GF; Genacryl Pink 3G; Genacryl Yellow 5GF; GFP (S65T); GFP red shifted (rsGFP); GFP wild type, non-UV excitation (wtGFP); GFP wild type, UV excitation (wtGFP); GFPuv; Gloxalic Acid; Granular Blue; Haematoporphyrin; Hoechst 33258; Hoechst 33342; Hoechst 34580; HPTS; Hydroxycoumarin; Hydroxystilbamidine (FluoroGold); Hydroxytryptamine; Indodicarbocyanine (DiD); Indotricarbocyanine (DiR); Intrawhite Cf; JC-1; JO-JO-1; JO-PRO-1; LaserPro; Laurodan; LDS 751; Leucophor PAF; Leucophor SF; Leucophor WS; Lissamine Rhodamine; Lissamine Rhodamine B; LOLO-1; LO-PRO-1; Lucifer Yellow; Mag Green; Magdala Red (Phloxin B); Magnesium Green; Magnesium Orange; Malachite Green; Marina Blue; Maxilon Brilliant Flavin 10 GFF; Maxilon Brilliant Flavin 8 GFF; Merocyanin; Methoxycoumarin; Mitotracker Green FM; Mitotracker Orange; Mitotracker Red; Mitramycin; Monobromobimane; Monobromobimane (mBBr-GSH); Monochlorobimane; MPS (Methyl Green Pyronine Stilbene); NBD; NBD Amine; Nile Red; Nitrobenzoxadidole; Noradrenaline; Nuclear Fast Red; Nuclear Yellow; Nylosan Brilliant lavin E8G; Oregon Green™; Oregon Green 488-X; Oregon Green™488; Oregon Green™ 500; Oregon Green™ 514; Pacific Blue; Pararosaniline (Feulgen); PE-Cy5; PE-Cy7; PerCP; PerCP-Cy5.5; PE-TexasRed (Red 613); Phloxin B (Magdala Red); Phorwite AR; Phorwite BKL; Phorwite Rev; Phorwite RPA; Phosphine 3R; PhotoResist; Phycoerythrin B [PE]; Phycoerythrin R [PE]; PKH26; PKH67; PMIA; Pontochrome Blue Black; POPO-1; POPO-3; PO-PRO-1; PO-PRO-3; Primuline; Procion Yellow; Propidium Iodid (PI); PyMPO; Pyrene; Pyronine; Pyronine B; Pyrozal Brilliant Flavin 7GF; QSY 7; Quinacrine Mustard; Resorufin; RH 414; Rhod-2; Rhodamine; Rhodamine 110; Rhodamine 123; Rhodamine 5 GLD; Rhodamine 6G; Rhodamine B 540; Rhodamine B 200; Rhodamine B extra; Rhodamine BB; Rhodamine BG; Rhodamine Green; Rhodamine Phallicidine; Rhodamine Phalloidine; Rhodamine Red; Rhodamine WT; Rose Bengal; R-phycoerythrin (PE); red shifted GFP (rsGFP, S65T); S65A; S65C; S65L; S65T; Sapphire GFP; Serotonin; Sevron Brilliant Red 2B; Sevron Brilliant Red 4G; Sevron Brilliant Red B; Sevron Orange; Sevron Yellow L; sgBFP™; sgBFP™(super glow BFP); sgGFP™; sgGFP™(super glow GFP); SITS; SITS (Primuline); SITS (Stilbene Isothiosulphonic Acid); SPQ (6-methoxy-N-(3-sulfopropyl)-quinolinium); Stilbene; Sulphorhodamine B can C; Sulphorhodamine G Extra; Tetracycline; Tetramethylrhodamine; Texas Red™; Texas Red-X™ conjugate; Thiadicarbocyanine (DiSC3); Thiazine Red R; Thiazole Orange; Thioflavin 5; Thioflavin S; Thioflavin TCN; Thiolyte; Thiozole Orange; Tinopol CBS (Calcofluor White); TMR; TO-PRO-1; TO-PRO-3; TO-PRO-5; TOTO-1; TOTO-3; TriColor (PE-Cy5); TRITC (TetramethylRodamineIsoThioCyanate); True Blue; TruRed; Ultralite; Uranine B; Uvitex SFC; wt GFP; WW 781; XL665; X-Rhodamine; XRITC; Xylene Orange; Y66F; Y66H; Y66W; Yellow GFP; YFP; YO-PRO-1; YO-PRO-3; YOYO-1; and YOYO-3. Methods for fluorophore labeling of nucleic acids are known in the art. For multiplex approaches, probes labeled with fluorophores with distinguishable excitation and/or emission spectra can be used.

### Generating Concatemers:

In various aspects, the underlying method of labeling a target-binding ligand comprises the generation of concatemers on the target-binding ligand, wherein the concatemeric repeat units provide multiple binding sites for nucleic acid probes on a single target-binding ligand molecule, thereby providing for many-fold amplification of signal from the target-binding ligand. As noted above, the methods, compositions and kits described herein permit multiplex labeling and detection of different target-binding ligands, achievable through the use of orthogonal concatemer/probe sets and the use of distinguishable labels. In one aspect, a concatemeric labeling method comprises, for example: (a) providing a conjugate of the target-binding ligand and a first oligonucleotide primer; (b) in a reaction mixture, contacting the conjugated first oligonucleotide primer with a first single-stranded extender template and a nucleic acid polymerase enzyme under conditions permitting hybridization and extension of the first oligonucleotide primer using the first single-stranded extender template; (c) heating the reaction mixture of step (b) to separate the first single-stranded extender template from extended first oligonucleotide primer produced in step (b); (d) repeating steps (b) and (c) at least once, thereby generating a concatemer comprising repeats of oligonucleotide primer sequence, conjugated to the target-binding ligand. In one embodiment of this and other methods described herein, the steps are performed in the order presented.

In various embodiments of the concatemeric labeling approach, the first single-stranded extender template comprises a concatemer of at least two head-to-tail copies of a sequence complementary to the first oligonucleotide primer, and a 3′ chain terminator, such that the extender template is not extended by the polymerase.

In various embodiments, the concatemer associated with a target-binding ligand can include branches that further expand the number of concatemeric repeats, and thereby expand the number of sites available to bind a probe molecule, thereby further increasing sensitivity. Thus, in such embodiments, the concatemeric labeling method further comprises, for example, after at least one repeat of steps (b)-(d), the steps of: (i) adding a second oligonucleotide primer and a second single-stranded extender template oligonucleotide, wherein the second oligonucleotide primer comprises, in 5′ to 3′ order, sequence complementary to the first oligonucleotide primer, and sequence complementary to the second single-stranded extender template, wherein the second single-stranded extender template comprises a 3′ chain terminator; (ii) heating the reaction mixture to separate nucleic acid strands and cooling to permit hybridization of the second oligonucleotide primer to concatemer generated in steps (b)-(d) and hybridization of the second single-stranded extender template to the second oligonucleotide primer; (iii) extending the second oligonucleotide primer using the second single-stranded extended template oligonucleotide as template; and (iv) repeating steps (ii) and (iii) at least once, thereby generating a concatemer comprising repeats of the second oligonucleotide primer. Additional rounds of branching can be performed with appropriate primer/extender combinations to even further expand the number of potential probe binding sites associated with a given target-binding ligand molecule.

In an alternative to generation of the branched concatemeric oligonucleotides, an approach can be used in which a first oligonucleotide primer is extended on a target-binding ligand bound to its target to generate a first concatemer as described herein, and pre-formed branched concatemers are then permitted to hybridize to a plurality of repeat elements on that first concatemer. In this alternative embodiment, the method further comprises, for example, after step (d): (i) adding, under conditions permitting hybridization, a nucleic acid comprising: (1) a second concatemer comprised of repeats of a second oligonucleotide sequence; and (2), a sequence complementary to the first oligonucleotide primer, such that a plurality of molecules of the nucleic acid comprising the second concatemer hybridizes via the sequence complementary to the first oligonucleotide primer to the concatemer formed in step (d). Additional rounds of branching concatemer addition can be performed to further increase the number of probe-binding sites associated with a given target-binding ligand.

The concatemer-generating or concatemer-labeling approaches described herein can be performed on a cell or tissue sample comprising or being assayed for target ligand. This has advantages in that the migration, diffusion and binding characteristics of the target-binding ligand are not strongly influenced by the labeling approach, since the target-binding ligand can already be bound to the cellular target—oligonucleotides of e.g., 40 nucleotides or less do not tend to significantly modify these characteristics of, e.g., antibodies as example target-binding ligands. Thus, while the first oligonucleotide primer can be longer or shorter as the case may be, in some embodiments the first oligonucleotide primer associated with the target binding ligand is on the order of 40 nucleotides or less, e.g., 35 nucleotides or less, 30 nucleotides or less, 25 nucleotides or less, 20 nucleotides or less, or 15 nucleotides or less.

The concatemer-generating approach for labeling can be performed in multiplex, such that a plurality of different target-binding ligands are labeled with linear or branched concatemers in the same set of reactions. An example of a method of multiplex labeling of a set of target ligands is set out in the following. In one approach, the method comprises: (a) providing a set of orthogonal first oligonucleotide primer and first single-stranded extender template oligonucleotide pairs, wherein the first oligonucleotide primer of each pair has a different sequence from other first oligonucleotide primers in the set, and is conjugated to a different member of a set of target-binding ligands; (b) in a reaction mixture, contacting the conjugated first oligonucleotide primers with the first single-stranded extender templates of the set of orthogonal pairs and a nucleic acid polymerase under conditions permitting hybridization and extension of the first oligonucleotide primers using the first single-stranded extender templates of the set of orthogonal pairs; (c) heating the reaction mixture of step (b) to separate the first single-stranded extender templates from extended first oligonucleotide primers produced in step (b); (d) repeating steps (b) and (c) at least once, thereby generating, on each member of the set of target ligands, a concatemer comprising repeats of oligonucleotide primer sequence, conjugated to the target-binding ligand.

In one embodiment, the first single-stranded extender template in each orthogonal first oligonucleotide primer/first single-stranded extender pair comprises a concatemer of at least two head-to-tail copies of a sequence complementary to the first oligonucleotide primer, and a 3′ chain terminator, such that the extender template is not extended by the polymerase.

In another embodiment, the steps of this and other methods set out herein are performed in the order presented.

The method described above provides multiplex linear concatemer generation. As discussed above, branched concatemers can further amplify signal for a target-binding ligand, and this approach is directly applicable in multiplex. Thus, a method that introduces branched concatemers in a multiplex reaction further comprises, for example, after step (d), the steps of: (i) adding a second set of orthogonal second oligonucleotide primer, second single-stranded extender template oligonucleotide pairs, wherein the second oligonucleotide primer in each pair comprises, in 5′ to 3′ order, sequence complementary to a member of the set of first oligonucleotide primers, and sequence complementary to the second single-stranded extender template of the second set of orthogonal oligonucleotide pairs, wherein each second single-stranded extender template comprises a 3′ chain terminator; (ii) heating the reaction mixture to separate nucleic acid strands and cooling to permit hybridization of the second oligonucleotide primers to concatemers generated in steps (b)-(d) and hybridization of the second single-stranded extender templates to the second oligonucleotide primers; (iii) extending the second oligonucleotide primers using the second single-stranded extended template oligonucleotides as template; and (iv) repeating steps (ii) and (iii) at least once, thereby generating, on respective members of the set of target-binding ligands, a concatemer comprising repeats of a respective second oligonucleotide primer. This approach provides branched concatemers in multiplex. Additional rounds of branching can be performed to further increase the signal from each target-binding ligand in the set.

Alternative approaches for concatemer generation and multiplex concatemer generation include the following: In one approach, is a method of generating a nucleic acid strand comprising concatemeric repeats of a given sequence comprises: a) providing in a reaction mixture, a first oligonucleotide primer and a first extender template oligonucleotide comprising a concatemer of at least two head-to-tail copies of sequence complementary to the first oligonucleotide primer, wherein the first extender template comprises a chain terminator at its 3′ end; b) incubating the reaction mixture under conditions permitting hybridization of the first oligonucleotide primer to the first extender template oligonucleotide; c) extending the hybridized first primer with a nucleic acid polymerase enzyme, thereby generating an extended nucleic acid strand complementary to the first extender template; d) heating the reaction mixture to separate the extended nucleic acid strand complementary to the first extender template from the first extender template; e) cooling the heated products to permit hybridization of the first extender template oligonucleotide to the extended nucleic acid strand complementary to the first extender template generated in step (c); f) extending the extended nucleic acid strand complementary to the first extender template generated in step (c) with the polymerase, thereby generating an extended nucleic acid strand comprising concatemeric repeats of the sequence comprised by the first oligonucleotide primer. In one embodiment, the method further comprises repeating steps (d) to (f) at least once, and preferably more, e.g., 2 times, 3 times, 5 times, 10 times, 15 times, 20 times, 30 times, 40times, 50 times, 60 times, 70 times, 80 times, 90 times, 100 times or more. In another embodiment, the steps are performed in the order presented. In another embodiment, the method comprises repeating steps (d) to (f) at least n times, wherein each iteration of steps (d) to (f) increases the concatemeric nucleic acid length by one concatemeric repeat.

Where multiplex is desired, step (a) can comprise providing a set of orthogonal first oligonucleotide primer and first extender template oligonucleotide pairs, such that steps (b)-(f) generate a multiplex set of concatemers, each comprising concatemeric repeats of the respective orthogonal oligonucleotide primer sequences.

Where branching can further increase signal, branching can also be applied in the context of this approach. Specifically, an adaptation of the method that further incorporates branching can further comprise, after one or more repetitions of steps (d) to (f): i) adding a second oligonucleotide primer and a second extender template oligonucleotide to the reaction, wherein the second oligonucleotide primer comprises a 5′ proximal sequence complementary to the first oligonucleotide primer sequence and a 3′ proximal sequence complementary to one of at least two repeat portions of the second extender template oligonucleotide, wherein the second extender template oligonucleotide comprises at least two head-to-tail repeats of sequence complementary to the second oligonucleotide primer and a chain terminator at its 3′ end; ii) hybridizing the second oligonucleotide primer to one or more concatemeric repeats on a concatemer formed after one or more repetitions of steps (d) to (f); iii) hybridizing the second extender template oligonucleotide to the second oligonucleotide primer; iv) extending the second oligonucleotide primer with the polymerase enzyme, thereby generating an extended nucleic acid strand complementary to the second extender template; v) heating the reaction mixture to separate the extended nucleic acid strand complementary to the second extender template from the second extender template; vi) cooling the heated products to permit hybridization of the second extender template oligonucleotide to the extended nucleic acid strand complementary to the second extender template generated in step (iv); vii) extending the extended nucleic acid strand complementary to the second extender template generated in step (iv) with the polymerase, thereby generating an extended nucleic acid strand comprising concatemeric repeats of the sequence comprised by the second oligonucleotide primer. In another embodiment, the method further comprises repeating steps (v) to (vii) at least once, and preferably more times, e.g., at least 2 times, 3 times, 5 times, 10 times, 15 times, 20 times, 30 times, 40 times, 50times, 60 times, 70 times, 80 times, 90 times, 100 times or more.

Where multiplex labeling and branching are desired, step (i) can comprise providing a set of orthogonal second oligonucleotide primer and second extender template oligonucleotide pairs, such that steps (ii)-(vii) generate a multiplex set of concatemers, each comprising concatemeric repeats of the respective orthogonal oligonucleotide primer sequences.

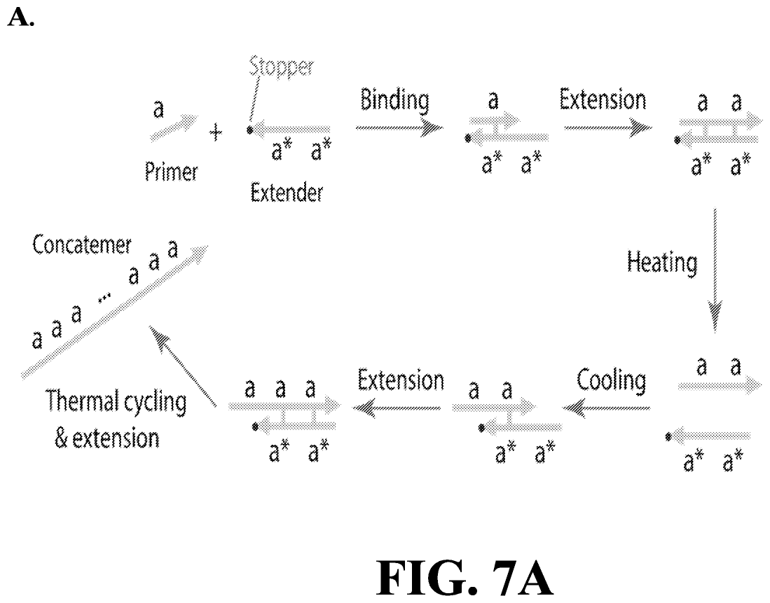

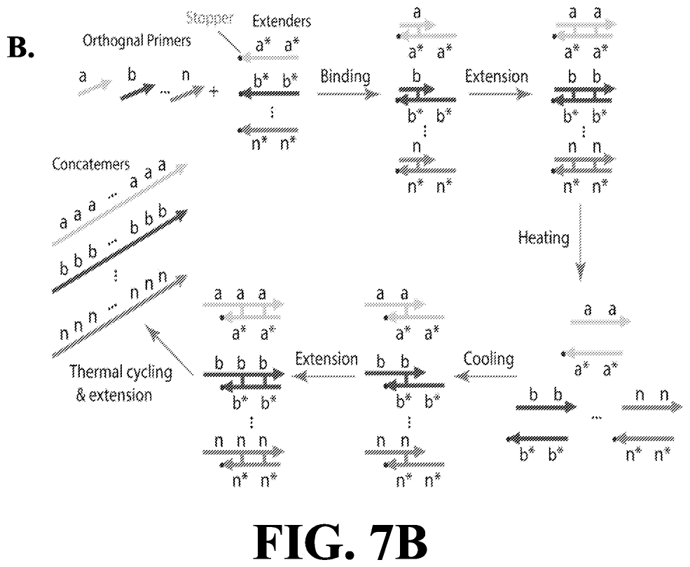

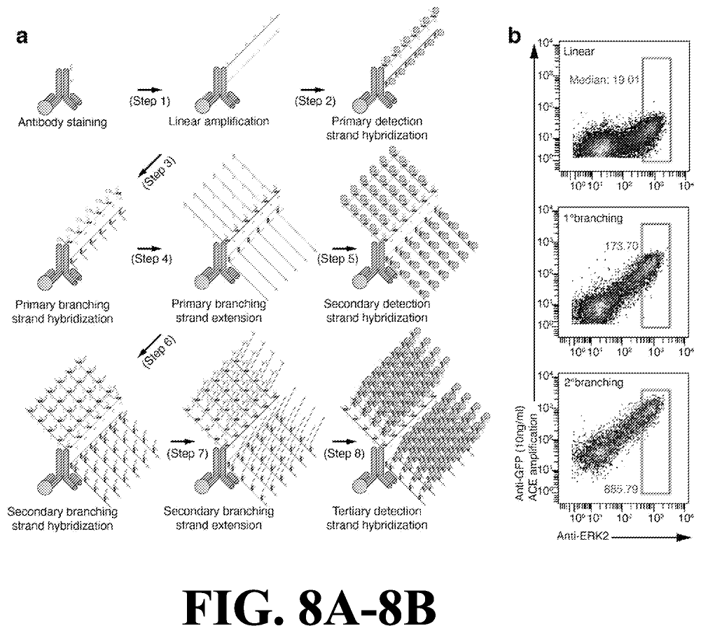

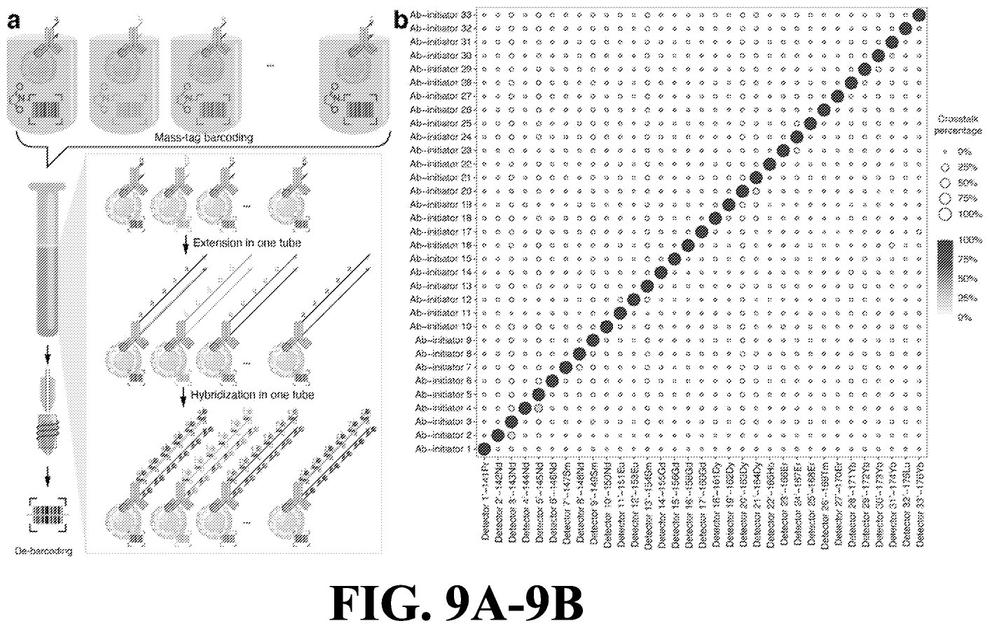

Several embodiments of concatemer-generating approaches described herein are illustrated in FIG. 7.

Single-stranded oligonucleotide primers will comprise, or consist of, a unit sequence that will be repeated in a concatemer generated using the methods described herein. The concatemer repeat element is represented in FIG. 7A as “a”. In effect, the actual concatemer repeat sequences will be determined by the sequence of the single-stranded extender template as described herein. That is, an oligonucleotide primer conjugated to a target-binding ligand can include 5′ sequence in addition to the sequence that becomes the concatemer repeat sequence, but the 3′ portion of the original oligonucleotide primer that is complementary to the sequence repeated in a head-to-tail manner in the single-stranded extender template will determine the repeat sequence. This may be illuminated by reference to, for example, FIG. 7A, which shows oligonucleotide Primer “a”, and Extender “a*a*,” in which a* is the complement of a. The optional inclusion of additional sequence (not shown) at the 5′ end of the Primer that is not necessarily complementary to a* would act as essentially a linker that does not get copied in the concatemer based on the extender template's copies of a *. Single-stranded extender templates are discussed in further detail herein below, and demonstrated in the Examples.

As discussed further herein below, an advantage of the methods, compositions and kits described herein is the ability to generate concatemer-labeled target-binding ligands (and ultimately to detect them) in multiplex. The length of the concatemer repeat sequence directly influences the degree of multiplex that can be achieved in the methods, compositions and kits described herein. Generally, the longer the repeat unit, the greater the degree of multiplexing one can achieve with orthogonal primers of that length. For example, for a repeat unit length of 9 nucleotides, a maximum of 93 orthogonal primers are possible. For a repeat unit length of 10 nt, the maximum increases to 170 primers. For a repeat unit length of 11 nt, the maximum is 315. For 12 nt, the maximum is 585. For 13 nt, the maximum is 1092. For 14 nt, the maximum is 2048, and for 15 nt, the maximum is 3855. For multiplex applications, it is preferred, but not absolutely required that the concatemer repeat unit on each target-binding ligand be the same length. Similar repeat unit lengths can provide, among other things, similar efficiencies for cycles of extender annealing and primer extension.

In one embodiment, the target molecule can itself be an RNA molecule, including but not limited to an mRNA molecule. The hybridization of one or more single-stranded oligonucleotides comprising a 5′ region complementary to a sequence on the target mRNA and a 3′ region comprising at least one sequence to be extended as a concatemer provides an initiation site for the generation of a concatemer using single-stranded extender template oligonucleotides as described herein. In this manner, a nucleic acid comprising this type of 5′ and 3′ regions can serve as a target-binding ligand molecule as that term is used herein. Where the target molecule is an RNA, the single-stranded target-binding ligand oligonucleotide can be hybridized to its RNA target in the cell, and then cross-linked so as to maintain its association with the target RNA through subsequent steps of concatemer generation. Cross-linking can be achieved, e.g., via the incorporation of photo cross-linking agents as described herein or as known in the art into the single-stranded target-binding ligand oligonucleotide (and preferably only into the portion of the oligonucleotide that hybridizes to target RNA). After hybridization and removal of non-hybridized oligonucleotide, the sample is irradiated with UV light to cross-link the oligonucleotide to its RNA target.

In one embodiment, the target molecule can itself be a DNA molecule, or a sequence on such a molecule, including, but not limited to a chromosomal or episomal DNA sequence. The hybridization of one or more single-stranded oligonucleotides comprising a 5′ region complementary to a sequence on the target DNA and a 3′ region comprising at least one sequence to be extended as a concatemer provides an initiation site for the generation of a concatemer using extender oligonucleotides as described herein. In this manner, a nucleic acid comprising this type of 5′ and 3′ regions can serve as a target-binding ligand molecule as that term is used herein. Where the target molecule is a DNA molecule of a given sequence, the single-stranded target-binding ligand oligonucleotide can be hybridized to its DNA target in the cell via target-complementary sequence located 5′ of the 3′ sequence to be extended as a concatemer repeat element, and then (after removal of non-hybridized oligonucleotide) cross-linked, e.g., as known in the art or as described herein, so as to maintain its association with the target DNA through subsequent steps of concatemer generation.

As noted, an important advantage of the methods, compositions and kits described herein is the ready ability to perform the concatemer-generating steps in multiplex, such that a plurality of different target-binding ligands have different concatemeric probe landing pads generated with the target-binding ligands associated with their cellular targets, in multiplex. This is illustrated, for example, in FIG. 7B, wherein orthogonal primers with repeat sequences “a,” “b” . . . “n” are extended using single-stranded extender templates with at least two repeats or copies of complementary sequences “a*,” “b*,” . . . “n*.” Once generated, the different concatemers provide landing pads for orthogonal complementary probes that can detect the various target ligands, also in multiplex. Various approaches to detecting and distinguishing probes in multiplex are known in the art and/or discussed elsewhere herein.

Target-binding ligands bearing oligonucleotide primers as described herein can be contacted with a cell or tissue sample preparation according to methods known in the art. Where, for example, the target-binding ligand is an antibody or antigen-binding fragment thereof, methods widely applied in immunohistochemistry can be used to stain the sample for detection of the given target ligand. Thus, the methods for contacting the cell or tissue sample with the target-binding ligand can parallel those used with, e.g., fluorescently labeled antibodies or antibody fragments. Where performed in multiplex, the contacting or staining can comprise the addition of a set of target-binding ligands, each comprising a different oligonucleotide primer. In instances where the target-binding ligand is not an antibody or antigen-binding fragment thereof, methods for contacting a cell or tissue preparation with the target-binding ligand can be adapted from those known in the art for the given ligand. In particular, where the target molecule is a DNA or RNA molecule comprising a given sequence, conditions as used, e.g., for in situ hybridization can be used to permit binding of the target-binding ligand oligonucleotide(s) to the target sequence(s). In some embodiments, it can be beneficial to remove unbound target-binding ligand prior to subsequent concatemer-generating or detection steps. This can be achieved, for example, by removal of target-binding ligand-containing solution from the cell or tissue sample, followed by one or more rinsing steps in an appropriate solution lacking the target-binding ligand.

### Generation of Concatemers:

In one embodiment, described herein is a method of labeling a target-binding ligand with a concatemer that provides sites for the binding of multiple copies of a labeled nucleic acid probe complementary to a concatemer repeat sequence. The following describes the generation of concatemers on target-binding ligands, and can be performed, for example, with the target-binding ligand bound to its target molecule in a cell sample. The methods generally use oligonucleotides conjugated to target-binding ligands, and single-stranded extender templates, with repeated cycles of annealing, polymerase extension and thermal strand separation, wherein each cycle adds another concatemeric repeat to the oligonucleotide conjugated to the target-binding ligand. In one embodiment, such a method of labeling a target-binding ligand comprises (a) providing a conjugate of the target-binding ligand and a first oligonucleotide primer; (b) in a reaction mixture, contacting the conjugated first oligonucleotide primer with a first single-stranded extender template and a nucleic acid polymerase enzyme under conditions permitting hybridization and extension of the first oligonucleotide primer using the first single-stranded extender template; (c) heating the reaction mixture of step (b) to separate the first single-stranded extender template from extended first oligonucleotide primer produced in step (b); and (d) repeating steps (b) and (c) at least once, thereby generating a concatemer comprising repeats of oligonucleotide primer sequence, conjugated to the target-binding ligand.

Conjugates of target-binding ligand and an oligonucleotide primer are discussed above. The exact makeup of reaction mixtures effective for the template-mediated extension of a primer, in terms of extender template and nucleotide concentrations, buffer, salts, divalent cations and other co-factors and their concentrations will depend upon the particular polymerase enzyme used, as will, for example, temperatures for the extension reactions. One of ordinary skill in the art can adjust these parameters for a given polymerase enzyme.

The melting temperature, Tₘ, for a nucleic acid duplex is a measure of thermal stability of the duplex, and defined as the temperature at which half of the DNA strands are in the single-stranded or dissociated state. The Tₘ for any given nucleic acid duplex is determined by parameters including its length, nucleotide composition, nucleic acid concentration and salt conditions in a given reaction. A rule of thumb formula for calculating Tₘ for short oligonucleotides is Tm=2×(A+T)+4×(G+C), but other formulae can provide more accurate estimates, e.g., Tm=81.5+0.41(% G+C)−675/N, where N is the total number of bases. Optimal annealing temperatures for primer extension reactions can be determined empirically by one of ordinary skill in the art, but will generally be about 5° C. below the Tₘ for a given primer/template duplex. When performed in multiplex, it is preferred that the annealing temperature be about 5° C. below the Tₘ for the primer/template duplex with the lowest Tm, but that primers be designed to minimize Tₘ differences. Primer design for multiplex primer extension reactions is well established in the art, and takes into account additional parameters, including, but not limited to minimizing primer-dimer formation (wherein one primer has sufficient complementarity to another primer in the same reaction to permit extension using the other primer as template) and minimizing off-target extension or amplification. Software tools for multiplex primer design are known in the art and include, for example, OligoPerfect™ Primer Designer, available from ThermoFisher Scientific, among others.

Thus, in a given concatemer-generating regimen, the oligonucleotide primer conjugated with a target-binding ligand, e.g., bound to its cellular target, is contacted in a reaction mixture appropriate for the polymerase enzyme used, with a single-stranded extender template oligonucleotide at a temperature, determined, for example, on the basis of Tₘ for the primer-template hybridized duplex. This permits annealing of single-stranded extender template to the primer, and extension of the annealed primer by the chosen polymerase enzyme when incubated at the extension temperature for the polymerase. Cycles of heating to dissociate the extended strand from the single-stranded extender template, cooling to permit annealing of single-stranded extender template and incubation at the extension temperature for the polymerase generate a concatemer with repeats of the oligonucleotide primer sequence defined by the single-stranded extender template. When performed in multiplex, concatemers of different, orthogonal repeat sequences on different target-binding ligands bound to their respective cellular targets are generated. Subsequent detection based on the hybridization of distinguishably-labeled probes to the concatemers permits detection, quantitation and/or imaging of the target molecules in or on the cell.

### Single-Stranded Extender Templates:

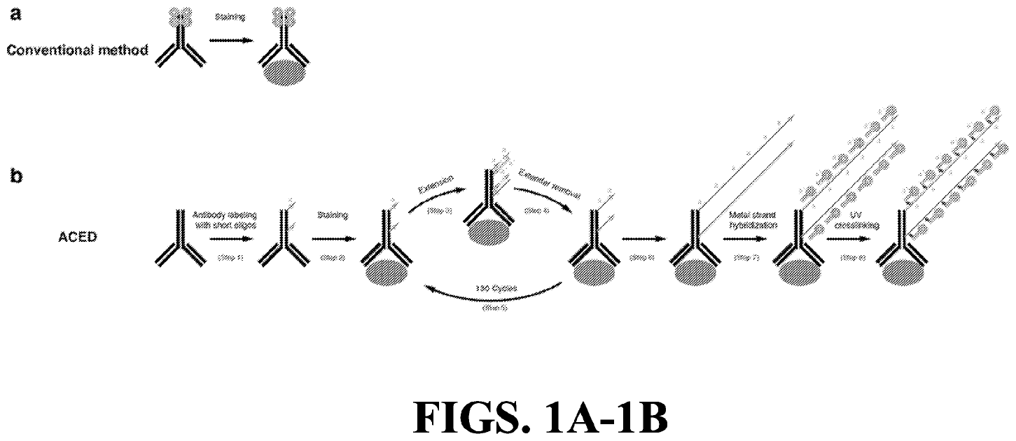

The use of single-stranded extender templates is central to the methods, compositions and kits described herein. Single-stranded extender templates useful in the methods, compositions and kits described herein comprise a concatemer of at least two head-to-tail copies of a sequence complementary to the oligonucleotide primer conjugated to the target-binding ligand. The single-stranded extender template further comprises a 3′ blocking or chain-terminating modification such that the single-stranded extender template is not itself extendable by the polymerase used to generate concatemers. The design of the single-stranded extender template provides for a method in which one copy of the concatemer repeat sequence complement hybridizes to the oligonucleotide conjugated to the target-binding ligand, and the at least one additional copy of the concatemer repeat sequence complement provides the template for the extension of the oligonucleotide conjugated to the target ligand. As a representative illustration, see again, e.g., FIG. 1A (Extender a*a*, with a 3′ “stopper” or chain terminator). Because the single-stranded extender template must include sequence complementary to the desired repeat sequence of a concatemer to be generated, the sequence of the single-stranded extender template is dictated by that desired repeat sequence. As discussed above in regard to primer design, the sequence of the desired concatemeric repeats is dictated by parameters permitting multiplex target detection while minimizing primer:primer interactions and off-target hybridization.

Where performed in multiplex, a set of different single-stranded extender templates, orthogonal to a set of oligonucleotide primers conjugated to respective members of a set of different target-binding ligands can be used. As with single-stranded extender templates used singly, each single-stranded extender template in a set comprises a concatemer of at least two head-to-tail copies of a sequence complementary to a different oligonucleotide primer conjugated to a target-binding ligand, e.g., as described herein. Each single-stranded extender template in such a set also comprises the 3′ blocking or chain-terminating modification that precludes polymerase extension of the extender template. Such a multiplex approach, illustrated, for example, in FIG. 1B (Extenders comprising a*a*, b*b* and n*n*, each with 3′ stoppers), generates concatemers with different repeat unit sequences on the respective members of the set of target-binding ligands.

The polymerase used to extend oligonucleotide primers and generate concatemers can be any of a number of template-dependent nucleic acid polymerases. In one embodiment, the polymerase is thermostable, such that it can withstand heating to a temperature and for a time sufficient to denature or dissociate double-stranded nucleic acids, retaining template-dependent polymerization activity when the reaction mixture is cooled to a temperature permitting annealing of extender template(s) to oligonucleotide primer(s) and primer extension. Different thermostable polymerases have different reaction buffer and extension temperature optima; these parameters are known to those of ordinary skill in the art and/or described in product literature for given polymerases. Non-limiting examples of thermostable polymerases useful in the methods, compositions and kits described herein include the following. Examples of polymerases that can be used in the methods described herein include but are not limited to: Standard Taq DNA polymerase (Cat. No. 10342053, Invitrogen, Carlsbad, CA), Platinum II Taq Hot-Start DNA Polymerase (Cat. No. 14966001, Invitrogen, Carlsbad, CA), Platinum SuperFi II DNA Polymerase (Cat. No. 12361010, Invitrogen, Carlsbad, CA), USB™ CycleSeq™ Thermostable DNA Polymerase (Cat. No. 792001000UN, Applied Biosystems, Waltham, MA); Taq DNA Polymerase (Cat. No. EP0402, ThermoScientific, Waltham, MA); HoTaq DNA Polymerase (HT-200, McLab, San Francisco, CA); I-5 Hi-Fi DNA Polymerase (PDP-100, McLab, San Francisco, CA); I-5 Hotstart DNA Polymerase (15HD-100, McLab, San Francisco, CA); DNA polymerase, thermotoga neapolitana (DPTN-100, McLab, San Francisco, CA); Pfu DNA Polymerase (AD-200, McLab, San Franscisco, CA); Pfu DNA Polymerase (Cat. No. 600135, Agilent Technologies, Wood Dale, IL); PfuTurbo DNA Polymerase (Cat. No. 600252, Agilent Technologies, Wood Dale, IL) and the like. One of ordinary skill in the art can identify additional polymerases that would function in the methods, compositions and kits described herein and can adjust reaction conditions as may be needed for any given polymerase.

### Branched Concatemers:

An option for further increasing the number of probe-binding sites on a target-binding ligand is to introduce branching such that initial, linear concatemers on a target-binding ligand provide sites for the generation of additional concatemers that branch off of the initial linear concatemers. It is important to note that in some embodiments, branching can be performed more than once, using earlier branches as sites for the generation of additional concatemers, with each round of branching multiplying the number of concatemer repeats and thus the number of potential probe-binding sites on a given target-binding ligand. In some embodiments, the branches are generated on a linear concatemer generated as described herein. In other embodiments, pre-formed concatemers are added to a reaction mixture that hybridize to repeats in a concatemer on a target ligand. These approaches are discussed further herein below.

In one embodiment, for the generation of branched concatemers on a target-binding ligand, after the generation of a linear concatemer as described herein, a second oligonucleotide primer and a second single-stranded extender template are added to the reaction mixture. Considerations for design of the second oligonucleotide primer for uniplex and for multiplex branched concatemer generation parallel those for design of the first oligonucleotide primer in terms of avoiding cross-talk, primer dimers and off-target hybridization. As with the first oligonucleotide primer, any of a number of different software packages can assist in such design.

In this embodiment, the second oligonucleotide primer comprises, in 5′ to 3′ order, sequence complementary to the first oligonucleotide primer, and sequence complementary to the second single-stranded extender template, wherein the second single-stranded extender template comprises a 3′ chain terminator. Molecules of the second oligonucleotide primer are permitted to anneal, via its sequence complementary to the first oligonucleotide primer, to at least one, and preferably a plurality of concatemeric repeats in the first extended concatemeric oligonucleotide conjugated to the target-binding ligand.

The sequence complementary to the first oligonucleotide primer can include a (photo) cross-linking moiety, such that after annealing, irradiation or other appropriate treatment of the reaction mixture will cross-link the second oligonucleotide primer to the extended first oligonucleotide primer so the second oligonucleotide primer remains associated with the extended first oligonucleotide primer through subsequent steps of thermal strand separation.

For concatemer generation, the second single-stranded extender template anneals to its complementary sequence on the second oligonucleotide primer, and the second oligonucleotide primer is extended, thereby adding a new copy of the second concatemeric repeat to the new branch from the first concatemer. Repeated cycling of thermal strand separation and second single-stranded template-directed primer extension generate concatemers comprising repeats including sequence in the second oligonucleotide primer. Where a plurality of second oligonucleotide primers hybridize to a plurality of concatemer repeats on the first extended concatemeric oligonucleotide, the first extended oligonucleotide provides, in effect, a stem with many branches, each branch including multiple repeats of the second concatemer repeat sequence. Additional rounds of branching with third, fourth, fifth or more oligonucleotide primers and their orthogonal single-stranded extender templates analogous in design to the second oligonucleotide primer/second single-stranded extender template can provide further amplification of the number of probe-binding sites associated with a given target-binding ligand.

An alternative branching approach generates concatemers of a second repeat sequence that comprise, e.g., at their 5′ ends, a sequence complementary to the first concatemer repeat. These second concatemeric molecules are then permitted to hybridize to repeats in the first target-binding ligand associated concatemer via that complementary sequence to provide the branched concatemers. The sequence complementary to the first concatemeric repeat can include a (photo) cross-linking moiety permitting cross-linking of the second concatemers to the first concatemer. It is contemplated that the second concatemeric molecules could themselves include branches with third, fourth, fifth or more repeat elements to further amplify the number of probe-binding sites associated with a given target-binding ligand.

Either of the alternative branching approaches discussed above can be used in multiplex reactions as described herein.

### Detection of Target Molecules:

Among other uses, the compositions, methods and kits described herein can be applied to the detection of target molecules in biological samples. Once concatemeric sequences are generated on a target-binding ligand bound to its target in or on a biological or cellular sample, a labeled nucleic acid probe (or set of orthogonal probes when multiplex detection is performed) can be contacted with and permitted to hybridize with concatemeric repeats on the target-binding ligand(s). Labeled probe molecules as described herein can include a cross-linking moiety that permits cross-linking of the probe to its complement. Labeled probe molecules are optionally cross-linked to their concatemeric sequences, e.g., via UV-activation of a cross-linking moiety and can then be detected in a manner appropriate for the given label, including but not limited to fluorescence-activated flow cytometry or cell sorting, and mass cytometry.

Thus, in various embodiments, detection comprises contacting a concatemer generated in prior steps with a labeled nucleic acid probe. In some embodiments, the nucleic acid probe comprises sequence complementary to a concatemer repeat. In some embodiments, the nucleic acid probe comprises sequence complementary to the oligonucleotide primer associated with a target-binding ligand. Where a branched concatemer is involved, repeats on the branched concatemer can be contacted with a nucleic acid probe complementary to those repeats. The hybridization of a plurality of nucleic acid probe molecules to a plurality of concatemeric repeats associated with a target-binding ligand provides for sensitive target detection and, when used in the multiplex permitted by the methods as disclosed, provides for the determination of expression profiles of 5 or more, 10 or more, 20 or more, 30 or more, 40 or more 50 or more, or as many as 60 or more different biological molecule targets at once.

Where multiple targets are detected in a single assay, the label on individual probes should be distinguishable from others used in the same assay. Where the labels are, for example, fluorescent, they should have excitation and/or emission spectra that permit the user to distinguish one from the other in the same assay. It is contemplated that repeated rounds of detection using different wavelengths for excitation of different fluorophores with non-overlapping excitation spectra can be performed. Similarly, detection of non-overlapping emission spectra from different fluorophores can be used to detect a plurality of different fluorophores associated with a plurality of different targets. It is also contemplated that a first round of fluorescently-labeled probes can be applied and detected, followed by photo-bleaching of the fluorophore, before application of a second round of different probes labeled with one or more of the same fluorophores as the first round.

Where mass cytometry is used for detection (see further below, and the Examples herein), each of the different metal ion labels present in or on a cell can be distinguished by mass to charge ratio, such that orthogonal probes bearing different metal ion labels can permit multiplex detection of different target molecules in the same assay.

Given the high degree of signal amplification provided by the concatemeric repeats on the target-binding ligands, detection down to as much as single-molecule sensitivity is achievable, in multiplex, using the methods described herein.

### Mass Cytometry:

Mass cytometry, also termed cytometry by Time-Of-Flight (CyTOF®), provides a tool for high-dimensional and high-throughput single-cell analyses. First introduced in 2009 (Bandura et al., Anal. Chem. 81: 6813-6822 (2009)), mass cytometry has become widely used in the analysis of immune cell function/activation and other processes due to its high-parameter capabilities. By using metal ion labels in place of fluorescent labels generally used in fluorescent-based flow cytometry, mass cytometry overcomes the problem of overlapping emission spectra that reduces the number of different targets or parameters that can be analyzed in a single assay.

Methods, applications and considerations for performing mass cytometry are reviewed, for example by Iyer et al., Front. Immunol. 13, Article 815828 (2022). Briefly, in mass cytometry, cells are generally incubated with a mixture of antibodies tagged with a unique non-radioactive heavy metal (most often, lanthanide) isotope. Single-cell suspensions are nebulized in a manner in which each droplet contains a single cell. Individual cells pass through argon (Ar) plasma, which atomizes and ionizes the sample. This process converts each cell into a cloud containing ions of the elements present in or on that cell. A high-pass optic (quadrupole) removes the low-mass-mainly biologic-ions from each cloud (i.e., those with mass below 75 Da), resulting in a cloud containing only those ions derived from the isotope-conjugated probes. In the Time of Flight (TOF) chamber, the ions are separated by mass-to-charge ratio. Upon encountering the detector, the ion counts are amplified and converted into electrical signals. Higher numbers of parameters corresponding to different targets are theoretically possible, but in current practice, about 60 different parameters are distinguishable in a mass cytometry panel. When adapted to use concatemer-based labeling/signal amplification methods as described herein, high multiplex, high sensitivity detection of a large number of even low-abundance targets can be performed.

As noted herein above, the concatemer-based signal amplification approaches described herein can be readily applied to other methods of biomolecule target detection or analysis. Non-limiting examples include imaging mass cytometry (see, e.g., Giesen et al., Nat. Methods 11: 417-422 (2014), see, also, the Hyperion+™ Imaging System (Fluidigm, Inc.; see the world wide web at fluidigm.com/products-services/technologies/imaging-mass-cytometry), multiplexed ion beam imaging (MIBI; see, e.g., Angelo et al. Nat. Med. 20: 436-442 (2014)), and multiplexed ion beam imaging by time of flight (MIBI TOF; see, e.g., Keren et al., Sci. Adv. 5: eaax5851 (2019)), among others.

It is noted that the concatemer-based methods and compositions described herein, and the cross-linking of probes with nucleic acids associated with target-binding ligands can be applied to or combined with other approaches for detecting target molecules. For example, the concatemer approaches described herein can be used to further amplify signal in RNAScope™(see, e.g., Wang, H. et al. (2015). Multiplex Fluorescent RNA In Situ Hybridization Via RNAscope. In: Hauptmann, G. (eds) In Situ Hybridization Methods. Neuromethods, vol 99 (2015). Humana Press, New York, NY). Another alternative is applicable to branched-DNA FISH or to InSituPlex™ (Ultivue) imaging. It is also noted that the cross-linking approaches described herein, including, but not limited to CNVK-mediated cross-linking, can be applied to these and other approaches to cross-link labeled probes to single or repeated nucleic acid sequences associated with a target molecule or with a target-binding ligand molecule. In such approaches, cross-linking of probe as described herein (e.g., using CNVK or other cross-linking agent as described herein conjugated with the probe) to nucleic acid associated with a molecule that hybridizes or binds to a given target RNA species, e.g., in RNAScope™ or branched-DNA FISH can be used to maintain the association of the probe with the nucleic acid associated with that molecule.

In addition to the concatemer-generating methods described herein above and demonstrated in the Examples herein, other methods of repeat generation can also be used to generate a plurality of probe-binding sites on a target molecule. Rolling Circle Amplification is a non-limiting example of such an additional method for the generation of multiple probe-binding sites on a target-binding ligand (see, e.g., Mohsen, M. G. et al. The Discovery of Rolling Circle Amplification and Rolling Circle Transcription. Acc. Chem. Res. 49: 2540-2550 (2016)). Such an approach can also be combined with cross-lining as described herein, including, but not limited to CNVK-mediated cross-linking, to attach probe molecules to repeated sequences in a manner that is stable to further processing steps that might otherwise dissociate the nucleic acid probe. Where it provides an advantage, the CNVK-mediated cross-linking can also be reversed (e.g., irradiation with a separate wavelength).

Signal amplification by exchange reaction (SABER; see, e.g., Treangen T. J. et al. Repetitive DNA and next-generation sequencing: computational challenges and solutions. Nat Rev Genet. 13: 36-46 (2011)) can also be adapted in a similar manner through use of the CNVK crosslinking chemistry. For example, long repeated sequences generated per the SABER approach can be hybridized and CNVK cross-linked to an oligonucleotide on a target-binding ligand (e.g., an antibody or other target-binding ligand), and nucleic acid probe molecules can be hybridized to and similarly CNVK cross-linked to those repeated sequences to provide signal amplification (e.g., in a manner in which the repeated sequences and probes do not dissociate from the target in subsequent steps). The same approaches can be applied where repeated sequences are generated, e.g., via hybridization chain reaction (HCR; See, e.g., Hybridization chain reaction: a versatile molecular tool for biosensing, bioimaging, and biomedicine. Chem. Soc. Rev. 46: 4281-4298 (2017).

In a further modification, the concatemer approach as described herein above and demonstrated in the Examples can be adapted to use click chemistry, instead of CNVK crosslinking, to covalently bind, e.g., a repeat-containing nucleic acid to a target-binding ligand and/or to covalently bind nucleic acid probe molecules to repeats on a target-binding ligand to amplify signal; see, e.g., ClampFISH (Tavakoli, S. et al. Chapter Twenty: Click-chemistry-based amplification and detection of endogenous RNA and DNA molecules in situ using clampFISH probes. 641: 459-476 (2020)).

### Ordered Extension and Nucleic Acid Generation

In another aspect, the single-stranded extender template approach can permit the generation of a nucleic acid having a desired sequence or ordered set of sequence elements. In this aspect, a set of different single-stranded extender elements can be used, each comprising sequence complementary to a prior extension element and a new extension element template. Repeated cycles of thermal strand separation and extension using successive single-stranded extension templates permits the generation of the nucleic acid having the desired sequence or ordered set of sequence elements.

In one embodiment, illustrated, for example, in FIG. 7C, an ordered extension approach to generating a nucleic acid molecule of a desired sequence comprises providing a first oligonucleotide primer, and, in a reaction mixture, contacting the first oligonucleotide primer with a first single-stranded extender template and a nucleic acid polymerase enzyme under conditions permitting hybridization and extension of the first oligonucleotide primer using the first single-stranded extender template. The polymerase can be a thermostable polymerase, e.g., as described herein or known in the art. The first single-stranded extender template comprises, in a 5′ to 3′ direction or orientation, a first extension template element, a sequence complementary to the first oligonucleotide primer, and a 3′ chain terminator. This step extends the first oligonucleotide primer by the complement of the first extension template element. It is noted that this ordered extension approach can be performed in which successive single-stranded extender templates are added at successive steps, or, in the alternative, a full set of single-stranded extender templates can be added at once, with successive thermal cycling building the ordered extension(s) in a single pot reaction based on specific hybridization of the appropriate single-stranded extender to each successively added extension.

Following the extension step, the mixture is heated to separate strands, and a second single-stranded extender template is contacted with the extended oligonucleotide primer generated in the first extension step, under conditions permitting hybridization and extension of the first extended oligonucleotide primer using the second single-stranded extender template. The second single-stranded extender template comprises, in a 5′ to 3′ direction or orientation, a second extension template element, a copy of the first extension template element, and a 3′ chain terminator. This step adds sequence complementary to the second extension template element to the first extended oligonucleotide primer. Successive rounds of strand separation and contacting with successive single-stranded extender templates, each including, in a 5′ to 3′ direction or orientation, a new extension template element, a copy of the previous extension template element, and a 3′ chain terminator can be performed. Design of the successive single-stranded extender templates permits the assembly of a nucleic acid molecule of essentially any desired sequence, comprised of sequence elements complementary to the successive extension template elements. Put differently, the ordered extension approach can be used to generate a nucleic acid molecule comprising, in order, first oligonucleotide primer sequence, then complement of each respective extension template element. If desired, among other uses, this approach can be used for barcoding a surface, a target-binding ligand, or other moiety to which the first oligonucleotide is conjugated.

The ordered extension approach can include, if desired, including a single-stranded extension template as described herein above for concatemer generation to the reaction such that one or more extension elements is repeated in concatemeric form in the generated nucleic acid molecule. This can be included before or after one or more rounds of single-stranded extender template switching that introduce new sequence elements to the growing extended oligonucleotide. Repeats added in this manner can thus provide for signal amplification as described herein.

The ordered extension approach can also be performed in multiplex. Primer design considerations are analogous to those for designing primers for multiplex concatemer-generating regimens described herein.

The ordered extension approach can be performed with the first oligonucleotide primer in solution, or on or conjugated to a surface upon which one wishes to build a nucleic acid molecule. The method can also be performed wherein the oligonucleotide primer is conjugated to a target-binding ligand as described herein. The method can also be performed in contact with a cell or tissue sample, such that the nucleic acid molecule is generated in situ in association with the cell or tissue sample, e.g., in association with a target molecule in the cell or tissue sample bound by a target-binding ligand.

The ordered extension approach can further comprise contacting the generated nucleic acid molecule with a labeled nucleic acid probe comprising sequence of one or more of the extension template elements. Detection of the label associated with the probe can provide information regarding the presence, amount and/or location of a target associated with the first oligonucleotide primer.

In an alternative approach, a method of adding a long DNA strand to extend only once, instead of short ones with multiple rounds of thermal cycling is also contemplated. The long DNA strand of one-step extension is a variation of the schematic of FIG. 1C—consider a2* to be repeats for imager binding. For the one-step extension scheme, instead of using heat to melt off the long DNA strand to allow imager binding and CNVK crosslinking, an alternative would be to incorporate uracil bases in the long DNA strand and use uracil DNA glycosylase enzyme (e.g., uracil DNA glycosylase; M0280S, New England Biolabs) digestion to fragment the long DNA strand after one-step extension to permit imager binding and CNVK crosslinking.

### Kits:

In various embodiments, a kit for performing one or more of the methods described herein is provided. Such kit can include, for example, a first oligonucleotide primer, or a set of first oligonucleotide primers as described herein, and reagents sufficient for conjugating the oligonucleotide primer to one or more target-binding ligands. A kit can include a target-binding ligand conjugated to an oligonucleotide primer or a set of different target-binding ligands conjugated to an orthogonal set of oligonucleotide primers. Target-binding ligands can include for example, peptides, polypeptides, nucleic acids, aptamers, a receptor and/or its cognate ligand, members of an affinity binding pair (including, but not limited to biotin/streptavidin), and small molecule agents that specifically bind a target molecule as that term is defined herein. Antibodies and antigen-binding fragments or constructs thereof represent one class of target-binding ligands useful in the methods, compositions and kits described herein. Nucleic acids comprising sequence complementary to a given target DNA or RNA molecule (including, but not limited to an mRNA molecule) represent another class of target-binding ligands that can be useful in the methods, compositions and kits described herein.

A kit can include a single-stranded extender template, or a set of single-stranded extender templates suitable for performing a concatemer-generating method as described herein.

Kits can include, e.g., orthogonal sets of oligonucleotide primers (conjugated to target-binding ligands or prepared so as to be so conjugated) and single-stranded extender templates permitting use in multiplex. Such orthogonal sets can be optimized to avoid primer/extender cross-talk, primer-dimer formation and off-target hybridization in multiplex labeling and/or detection reactions. Kits can also include a polymerase, e.g., a thermostable polymerase as described herein or as known in the art, as well as nucleotides and reaction/buffer components suitable for primer extension using the given polymerase enzyme. Kits can also further include one or more labeled nucleic acid probe molecules and/or reagents for labeling a nucleic acid probe molecule. The probe molecules can be complementary to, e.g., a concatemer repeat element, or to one or more elements in an ordered extension product generated as described herein. The labeled nucleic acid probe molecules can comprise a cross-linking moiety. The cross-linking moiety can be a UV cross-linking moiety, and can include, for example, 3-cyanovinylcarbazole phosphoramidite (CNvK). Labels can include any label described herein or known in the art, including, but not limited to fluorescent labels, metal ion labels, etc. The metal ion label can be, for example, a lanthanide metal ion, or can include, for example, cadmium, palladium, indium, platinum, bismuth, selenium, tellurium, gold, silver and tantalum, yttrium, ruthenium, rhodium, iodine, osmium and iridium, among others. In some embodiments, the labeled nucleic acid probe or probes can contain a metal ion label and a fluorescent label.

Kits can include packaging materials for the various components and, e.g., instructions for use.

The technology may be as described in any one of the following numbered Embodiments:

Embodiment 1: A method of detecting a target molecule in mass cytometry, the method comprising: a) contacting a target-binding ligand molecule comprising a conjugated oligonucleotide primer with a cell or tissue sample under conditions permitting binding of the target-binding ligand molecule to its target in the cell or tissue sample; b) extending the oligonucleotide primer to comprise a concatemer of sequence comprised by the oligonucleotide primer; c) contacting the extended oligonucleotide primer of step (b) with a labeled probe nucleic acid comprising sequence complementary to sequence repeated in the concatemer, such that a plurality of labeled probe nucleic acid molecules hybridizes to a plurality of sequence repeats in the concatemer; d) cross-linking the hybridized probe nucleic acid molecules to the extended oligonucleotide primer; and e) detecting labeled probe using mass cytometry.

Embodiment 2: The method of Embodiment 1, wherein the labeled probe nucleic acid comprises a metal ion label.

Embodiment 3: The method of Embodiment 1 or Embodiment 2, wherein the labeled probe comprises a fluorophore.

Embodiment 4: The method of any one of Embodiments 1-3, wherein extending the oligonucleotide primer comprises thermal cycling with a single-stranded extender template and a template-dependent nucleic acid polymerase enzyme.

Embodiment 5: The method of Embodiment 4, wherein the polymerase enzyme is thermostable.

Embodiment 6: The method of Embodiment 4 or Embodiment 5, wherein the single-stranded extender template comprises a concatemer of at least two head-to-tail copies of a sequence complementary to the oligonucleotide primer, and a 3′ chain terminator, such that the extender template is not extended by the polymerase.

Embodiment 7: The method of any one of Embodiments 1-6, wherein extending step (b) comprises a plurality of cycles of extension, thermal strand separation, and cooling to permit single-stranded extender annealing and extension by the polymerase, whereby successive cycles add successive concatemeric repeats to the oligonucleotide primer. Embodiment 8: The method of any one of Embodiments 1-7, performed in multiplex with a plurality of different target-binding ligand molecules, orthogonal sets of single-stranded extender templates, and distinguishably labeled orthogonal probe nucleic acids.

Embodiment 9: The method of any one of Embodiments 1-8, wherein the concatemer comprises a branched concatemer, formed on the target-binding ligand via thermal cycling.

Embodiment 10: The method of Embodiment 3, further comprising the step, before detection step (e), of imaging fluorophore associated with the cell or tissue sample.

Embodiment 11: The method of any one of Embodiments 1-10, wherein cross-linking the hybridized probe nucleic acid molecules to the extended oligonucleotide primer comprises photo cross-linking.

Embodiment 12: The method of Embodiment 11, wherein the probe nucleic acid molecules comprise a photo cross-linking agent.

Embodiment 13: The method of Embodiment 12, wherein the photo cross-linking agent comprises 3-cyanovinylcarbazole phosphoramidite (CNVK), and cross-linking comprises UV irradiation.

Embodiment 14: The method of any one of Embodiments 1-13, wherein the target-binding ligand comprises an antibody or antigen-binding fragment thereof.

Embodiment 15: A method of fluorescence-activated cell sorting, the method comprising: a) providing a sample comprising fixed cells; b) contacting a target-binding ligand molecule comprising a conjugated oligonucleotide primer with cells in the sample under conditions permitting binding of the target-binding ligand molecule to its target in or on the cells; c) extending the oligonucleotide primer to comprise a concatemer of sequence comprised by the oligonucleotide primer; d) contacting the extended oligonucleotide primer of step (c) with a fluorescently labeled probe nucleic acid comprising sequence complementary to sequence repeated in the concatemer, such that a plurality of labeled probe nucleic acid molecules hybridizes to a plurality of sequence repeats in the concatemer; e) cross-linking the hybridized probe nucleic acid molecules to the extended oligonucleotide primer; and f) sorting the cells via fluorescence-activated cell sorting.

Embodiment 16: The method of Embodiment 15, wherein the labeled probe further comprises a metal ion.

Embodiment 17: The method of Embodiment 15 or 16, wherein extending the oligonucleotide primer comprises thermal cycling with a single-stranded extender template and a template-dependent nucleic acid polymerase enzyme.

Embodiment 18: The method of Embodiment 17, wherein the polymerase enzyme is thermostable.

Embodiment 19: The method of Embodiment 17 or Embodiment 18, wherein the single-stranded extender template comprises a concatemer of at least two head-to-tail copies of a sequence complementary to the oligonucleotide primer, and a 3′ chain terminator, such that the extender template is not extended by the polymerase.

Embodiment 20: The method of any one of Embodiments 15-19, wherein extending step (c) comprises a plurality of cycles of extension, thermal strand separation, and cooling to permit single-stranded extender annealing and extension by the polymerase, whereby successive cycles add successive concatemeric repeats to the oligonucleotide primer.

Embodiment 21: The method of any one of Embodiments 15-20, performed in multiplex with a plurality of different target-binding ligand molecules, orthogonal sets of single-stranded extender templates, and distinguishably labeled orthogonal probe nucleic acids.

Embodiment 22: The method of any one of Embodiments 15-21, wherein the concatemer comprises a branched concatemer, formed on the target-binding ligand via thermal cycling.

Embodiment 23: The method of any one of Embodiments 16-22, further comprising the step, after sorting step (f), of detecting metal ion-labeled probe via mass cytometry.

Embodiment 24: The method of any one of Embodiments 15-23, wherein cross-linking the hybridized probe nucleic acid molecules to the extended oligonucleotide primer comprises photo cross-linking.

Embodiment 25: The method of any one of Embodiments 15-24, wherein the probe nucleic acid molecules comprise a photo cross-linking moiety.

Embodiment 26: The method of Embodiment 25, wherein the photo cross-linking moiety comprises 3-cyanovinylcarbazole phosphoramidite (CNVK), and cross-linking comprises UV irradiation.

Embodiment 27: The method of any one of Embodiments 15-26, wherein the target-binding ligand comprises an antibody or antigen-binding fragment thereof.

Embodiment 28: A composition comprising a target-binding ligand and a concatemeric oligonucleotide conjugated to the target-binding ligand.

Embodiment 29: The composition of Embodiment 28, wherein the concatemeric oligonucleotide further comprises one or more branched concatemeric oligonucleotides.

Embodiment 30: The composition of Embodiment 28, further comprising a plurality of nucleic acid probe molecules hybridized to concatemeric repeats of the concatemeric oligonucleotide.

Embodiment 31: The composition of Embodiment 30, wherein the nucleic acid probe molecules comprise a cross-linking moiety.

Embodiment 32: The composition of Embodiment 31, wherein the cross-linking moiety comprises 3-cyanovinylcarbazole phosphoramidite (CNVK).

Embodiment 33: The composition of Embodiment 31 or 32, wherein the nucleic acid probe molecules are cross-linked to the concatemeric repeats.

Embodiment 34: The composition of any one of Embodiments 30-33, wherein the nucleic acid probe molecules comprise a fluorescent label or a metal ion label.

Embodiment 35: The composition of any one of Embodiments 30-34, wherein the nucleic acid probe molecules comprise a fluorescent label and a metal ion label.

Embodiment 36: The composition of any one of Embodiments 30-35, wherein the target-binding ligand comprises an antibody or antigen-binding fragment thereof.

Embodiment 37: A cell or tissue sample comprising a composition of any one of Embodiments 30-36, wherein the target-binding ligand is bound to a target molecule in or on the cell or tissue sample.

Embodiment 38: A kit for performing one or more of the methods as described herein, the kit comprising: a) a target-binding ligand conjugated to an oligonucleotide primer; or a set of target-binding ligands conjugated to orthogonal oligonucleotide primers; b) a single-stranded extender template or a set of orthogonal single-stranded extender templates; c) a labeled probe nucleic acid or a set of orthogonal labeled probe nucleic acids, wherein the labeled probe nucleic acid or set of orthogonal labeled probe nucleic acids comprises a cross-linking moiety.

Embodiment 39: The kit of Embodiment 38, wherein each single-stranded extender template comprises a concatemer of at least two head-to-tail copies of a sequence complementary to the oligonucleotide primer, and a 3′ chain terminator, such that the extender template is not extended by a polymerase.

Embodiment 40: The kit of Embodiment 38, further comprising a template-dependent nucleic acid polymerase enzyme.

Embodiment 41: The kit of Embodiment 40, wherein the polymerase enzyme is thermostable.

Embodiment 42: The kit of any one of Embodiments 38-41, wherein the target-binding ligand comprises an antibody or antigen-binding fragment thereof.

Embodiment 43: The kit of any one of Embodiments 38-42, wherein the orthogonal nucleotide primers, single stranded extender templates and nucleic acid probes are optimized to avoid primer/extender cross-talk, primer-dimer formation, and off-target hybridization.

Embodiment 44: The kit of any one of Embodiments 38-43, wherein the single-stranded extender template or the set of single-stranded extender templates are suitable for performing a concatemer-generating method.

Embodiment 45: The kit of any one of Embodiments 38-44, further comprising packaging materials for the various components and instructions for use.

Embodiment 46: The kit of any one of Embodiments 38-45, further comprising one or more target-binding ligand molecules or reagents for conjugating a first oligonucleotide to a target-binding ligand.

Embodiment 47: The kit of any one of Embodiments 38-46, further comprising a thermostable polymerase, nucleotides, reaction buffer components, and reagents for labeling a nucleic acid probe molecule.

Embodiment 48: The kit of any one of Embodiments 38-47, wherein the probe molecule can be complementary to a concatemer repeat element.

Embodiment 49: The kit of any one of Embodiments 38-48, wherein the cross-linking moiety comprises a photo cross-linking moiety.

Embodiment 50: The kit of any one of Embodiments 38-49, wherein the cross-linking moiety comprises 3-cyanovinylcarbazole phosphoramidite (CNVK).

Embodiment 51: The kit of any one of Embodiments 38-50, wherein the labeled probe nucleic acid or set of orthogonal labeled probe nucleic acids comprises a fluorescent label or a metal ion label.

Embodiment 52: The kit of any one of Embodiments 38-51, wherein the labeled nucleic acid probe molecules comprise a fluorescent label and a metal ion label.

Specific elements of any of the disclosed embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

### EXAMPLES

The following examples illustrate some embodiments and aspects of the invention. It will be apparent to those skilled in the relevant art that various modifications, additions, substitutions, and the like can be performed without altering the spirit or scope of the invention. and such modifications and variations are encompassed within the scope of the invention as defined in the claims which follow. The following examples do not in any way limit the invention.

### Example 1

Mass cytometry, a recent established approach based on inductively coupled plasma time-of-flight mass spectrometry and a single-cell sample introduction system, allows simultaneous quantification of>50 proteins or protein modifications at single-cell resolution, enabling the profiling of complex cellular behaviors in highly heterogeneous samples. In mass cytometry, metal isotope-tagged antibodies are used to label proteins or protein modifications in cells. During the sample acquisition, each stained single cell is vaporized, atomized, and ionized. The metals in the formed ion cloud are quantitatively analyzed by the mass spectrometer to yield a high-dimensional single-cell proteomic readout. Previous researchers have introduced mass cytometry as a versatile approach to assess the signaling network states of over 30 phosphorylation sites in millions of single cells. Relationships between all pairs of measured phosphorylation sites can be computed to infer network responses to a stimulus or to trace the network reshaping through a phenotypical transition. In combination with high-throughput screening assays, these types of experiments have revealed novel signaling mechanisms involved in cancer progression and drug resistance.

However, single-cell signaling network analysis has not been transformed to study patient-derived tumor samples in clinical cancer research. Similar to many other type of single cell approaches, mass cytometry faces a sensitivity limitation that˜300 metal tagged antibody targeting the same epitope has to present in one cell to generate a detectable signal. For single-cell signaling network profiling in cancer samples, the sensitivity limitation has an even higher impact as the basal levels of phosphorylation sites (i.e., without additional stimulation) typically do not reach the detection limit for mass cytometry, especially in cells of smaller volume, such as infiltrated T cells in a tumor sample. In addition, phosphorylated residues are difficult to be fully preserved in tissue samples. Factors such as temperature fluctuation and fixation protocols may further reduce the phosphorylation levels before samples can be analyzed. In the imaging mode of mass cytometry analysis (IMC), the imaging resolution has to be compromised in compensation of low sensitivity that the spatial cues determining a signaling outcome is difficult to be identified.

A DNA nanodevice was recently created that undergoes repeated in situ concatenation in thermocycling conditions. Combining this device with a newly developed photo-crosslinking strategy based on 3-cyanovinylcarbazole phosphoramidite (CNvK) modification, the method has been successfully implemented to amplify the mass cytometry signal to address its sensitivity bottleneck. This has allowed comprehending cell state and predicting cell fate in biological or clinical samples.

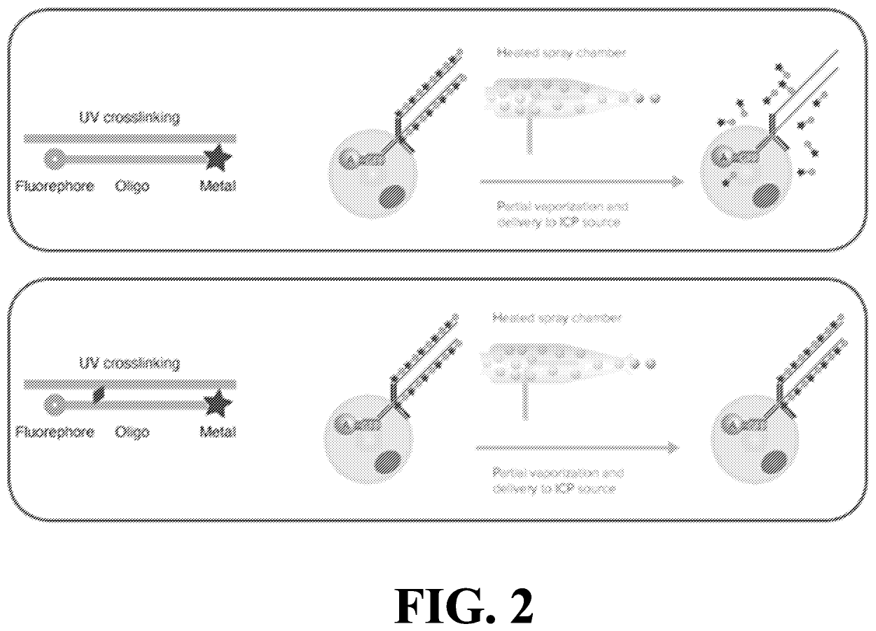

Method. In the conventional mass cytometry method, metal isotopes are first chelated into a maleimide-modified diethylenetriamine pentaacetate (DTPA) polymer that is subsequently conjugated to the reactive cysteine residues located on the hinge region of a partially reduced antibody. With this approach, only a few modification sites presenting on each antibody can be conjugated, carrying limited number of metal ions (FIG. 1A). To increase the sensitivity of mass cytometric analysis, the main innovation of my novel approach, termed amplification by cyclic extension of DNA oligo (ACED), is to engineer DNA nano-devices that create unlimited repeats of metal probe hybridization sites in situ (FIG. 1B). In ACED, antibodies targeting the protein of interest were conjugated with short DNA oligo initiators (TT-a, 11-mer) that maintains antibody binding affinity, maximizes antibody diffusion capability intracellularly, and reduces nonspecific bindings that are often seen in methods applying long oligo (>40-mer) conjugates 14,15 (FIG. 1B, step 1). Conjugated antibodies are mixed in a staining solution and applied on cell suspensions for cell surface or intracellular marker staining (step 2). Next, an extender with two complementary repeats of the initiator sequence (a′-T-a′, 19-mer) is introduced to the stained cells. At low temperature, the extender and initiator hybridize to allow BST polymerase-medicated strand extension (forming TT-a-A-a, step 3). By increasing the reaction temperature, extenders are removed to expose the single-stranded extended probe (step 4). The thermal cycles are then repeated in a desired number of rounds to successively elongate the probe conjugated to the antibody (step 5) that creates hundreds of a-A repeats on each antibody modification site (step 6). DTPA polymers with chelated Ln3⁺ metal ions are conjugated to detection strands with the sequence of a′-T-a′ through maleimide-thiol reaction that can subsequently hybridize to the extended DNA probes on an antibody (step 7), each occupying one of metal probe binding site (a-A-a). A short-time (1 second) ultraviolet (UV) light exposure activates the 3-cyanovinylcarbazole phosphoramidite (CNVK) photo-cross-linker on the metal probe strand that creates covalent binding between the hybridized DNA molecules and allows DTPA polymers to be attached to the antibody (step 8) (FIG. 2).

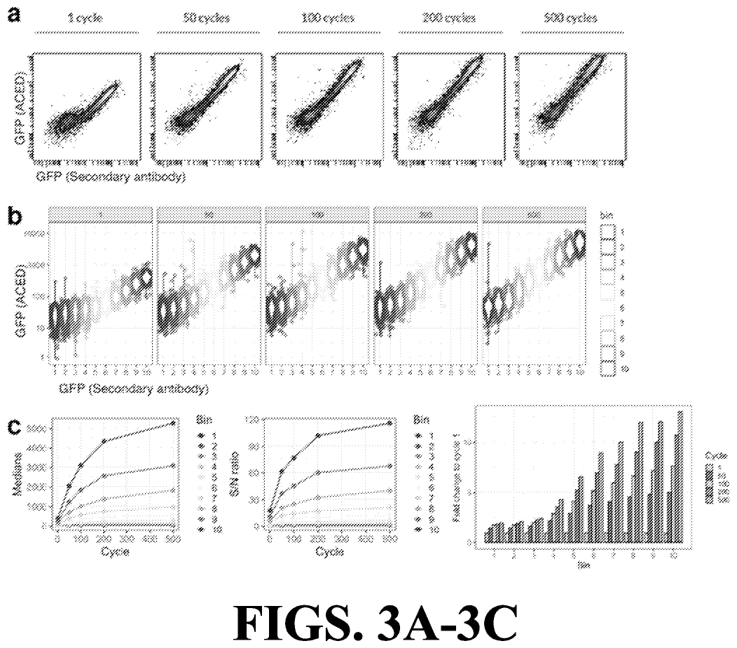

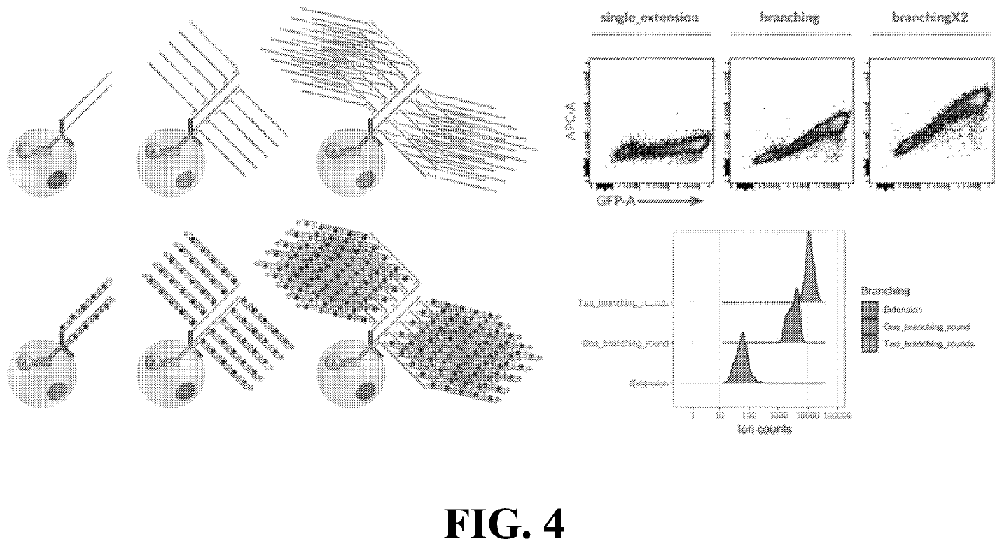

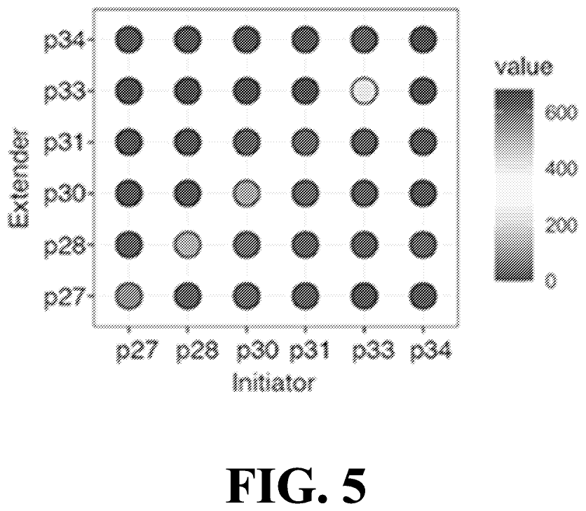

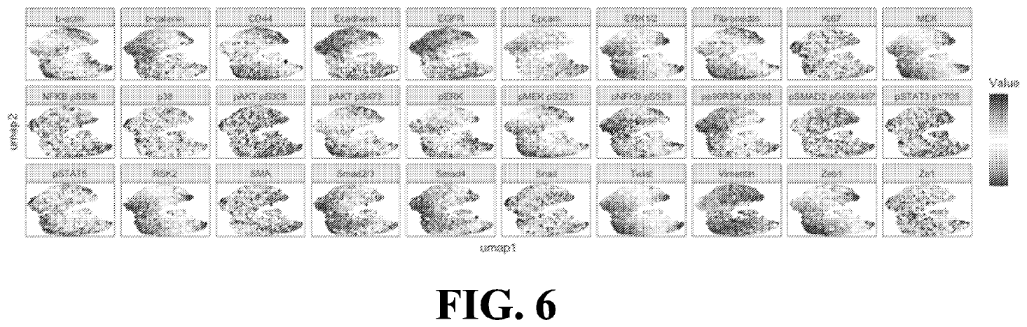

To validate the specificity and to quantify the amplification power of ACED on mass cytometric analysis, this system was applied to the human embryonic kidney HEK293T cells that transiently overexpress green fluorescent protein GFP with a high expression gradient. As expected, ACED-amplified signal of GFP antibody largely correlated with the secondary antibody signal targeting the primary GFP antibody, confirming the specificity of the established DNA device in intracellular antibody signal amplification (FIG. 3A). Through a time-series analysis over 500 thermo cycles, we showed that the ACED approach consistently amplified mass cytometry signal over the thermocycling time course (FIG. 3A). Quantification using the binned data according to GFP expression level (FIG. 3B), a 13-fold amplification strength and a six-fold signal-to-noise ratio enhancement were observed in samples with 500 rounds amplification compared to the unamplified sample (FIG. 3C). To detect and quantify ultra-low abundance proteins, linear ACED amplification might be inefficient and time-consuming. For these proteins, we developed a branching amplification strategy that, through iterative rounds of ACED reaction, the amplification power can be exponentially increased. Quantifying the branching results from mass cytometry analysis, a 17-fold amplification for each branching round could be observed (FIG. 4). Ultimately, unlimited signal amplification can be achieved with multiple branching reactions. To multiplex the ACED method high orthogonality between DNA probes targeting different antibodies is required. 50 orthogonal ACED barcode sequences were designed that covers the full capacity of mass cytometry. In a proof-of-concept test, six initiator probes were cross reacted with six extender probes to thoroughly assess their orthogonality (FIG. 5). Only the matching pairs of initiator and extender sequence generated detectable signal in the follow-up mass cytometry analysis (FIG. 5). The 30-plex signal amplification was then performed on a mouse Py2T cell line that underwent epithelial-to-mesenchymal transition (EMT) during a 7-day time course. With ACED, key signature molecules including E-cadherin, vimentin, Smad2/3, Smad4, and most importantly, the transcriptional factors Snail/Slug, Zeb1, could be detected, differentiated and quantified during the progression of the EMT process (FIG. 6). A dimensional reduction analysis using uniform manifold approximation and projection (UMAP) 16 projected the EMT progression trajectory through the time course that validated ACED as a robust technological platform to precisely amplify mass cytometry signal, particularly for low abundance proteins (FIG. 6).

### REFERENCES

1. Bandura, D. R., Baranov, V. I., Ornatsky, O. I., Antonov, A., Kinach, R., Lou, X., Pavlov, S., Vorobiev, S., Dick, J. E., & Tanner, S. D. Mass cytometry: technique for real time single cell multitarget immunoassay based on inductively coupled plasma time-of-flight mass spectrometry. Anal. Chem. 81, 6813-22 (2009) PMID: 19601617.
2. Bendall, S. C., Simonds, E. F., Qiu, P., Amir, E. D., Krutzik, P. O., Finck, R., Bruggner, R. V, Melamed, R., Trejo, A., Ornatsky, O. I., Balderas, R. S., Plevritis, S. K., Sachs, K., Pe'er, D., Tanner, S. D., & Nolan, G. P. Single-cell mass cytometry of differential immune and drug responses across a human hematopoietic continuum. Science 332, 687-96 (2011) PMID: 21551058.
3. Bodenmiller, B., Zunder, E. R., Finck, R., Chen, T. J., Savig, E. S., Bruggner, R. V, Simonds, E. F., Bendall, S. C., Sachs, K., Krutzik, P. O., & Nolan, G. P. Multiplexed mass cytometry profiling of cellular states perturbed by small-molecule regulators. Nat. Biotechnol. 30, 858-67 (2012) PMID: 22902532.
4. Chevrier, S., Levine, J. H., Zanotelli, V. R. T., Silina, K., Schulz, D., Bacac, M., Ries, C. H., Ailles, L., Jewett, M. A. S., Moch, H., van den Broek, M., Beisel, C., Stadler, M. B., Gedye, C., Reis, B., Pe'er, D., & Bodenmiller, B. An Immune Atlas of Clear Cell Renal Cell Carcinoma. Cell 169, 736-749.e18 (2017) PMID: 28475899.
5. Levine, J. H., Simonds, E. F., Bendall, S. C., Davis, K. L., Amir, E. D., Tadmor, M. D., Litvin, O., Fienberg, H. G., Jager, A., Zunder, E. R., Finck, R., Gedman, A. L., Radtke, I., Downing, J. R., Pe'er, D., & Nolan, G. P. Data-Driven Phenotypic Dissection of AML Reveals Progenitor-like Cells that Correlate with Prognosis. Cell 162, 184-197 (2015) PMID: 26095251.
6. Lun, X. K., Zanotelli, V. R. T., Wade, J. D., Schapiro, D., Tognetti, M., Dobberstein, N., & Bodenmiller, B. Influence of node abundance on signaling network state and dynamics analyzed by mass cytometry. Nat. Biotechnol. 35, 164-172 (2017) PMID: 28092656.
7. Lun, X.-K., Szklarczyk, D., Gábor, A., Dobberstein, N., Zanotelli, V. R. T., Saez-Rodriguez, J., von Mering, C., & Bodenmiller, B. Analysis of the Human Kinome and Phosphatome by Mass Cytometry Reveals Overexpression-Induced Effects on Cancer-Related Signaling. Mol. Cell (2019) doi:10.1016/J.MOLCEL.2019.04.021 PMID: 31101498.
8. Wade, J. D., Lun, X.-K., Zivanovic, N., Voit, E. O., & Bodenmiller, B. Mechanistic Model of Signaling Dynamics Across an Epithelial Mesenchymal Transition. Front. Physiol. 11, (2020) PMID: 33329028.
9. Krishnaswamy, S., Zivanovic, N., Sharma, R., Pe'er, D., & Bodenmiller, B. Learning time-varying information flow from single-cell epithelial to mesenchymal transition data. PLoS One 13, e0203389 (2018) PMID: 30372433.
10. Krishnaswamy, S., Spitzer, M. H., Mingueneau, M., Bendall, S. C., Litvin, O., Stone, E., Pe'er, D., & Nolan, G. P. Conditional density-based analysis of T cell signaling in single-cell data. Science science. 1250689-(2014) doi:10.1126/science.1250689 PMID: 25342659.
11. Giesen, C., Wang, H. A. O., Schapiro, D., Zivanovic, N., Jacobs, A., Hattendorf, B., Schüffler, P. J., Grolimund, D., Buhmann, J. M., Brandt, S., Varga, Z., Wild, P. J., Günther, D., & Bodenmiller, B. Highly multiplexed imaging of tumor tissues with subcellular resolution by mass cytometry. Nat. Methods 11, 417-422 (2014) PMID: 24584193.
12. Jackson, H. W., Fischer, J. R., Zanotelli, V. R. T., Ali, H. R., Mechera, R., Soysal, S. D., Moch, H., Muenst, S., Varga, Z., Weber, W. P., & Bodenmiller, B. The single-cell pathology landscape of breast cancer. Nature 578, 615-620 (2020) PMID: 31959985.
13. Schulz, D., Zanotelli, V. R. T., Fischer, J. R., Schapiro, D., Engler, S., Lun, X.-K., Jackson, H. W., & Bodenmiller, B. Simultaneous Multiplexed Imaging of mRNA and Proteins with Subcellular Resolution in Breast Cancer Tissue Samples by Mass Cytometry. Cell Syst. 6, 25-36.e5 (2018) PMID: 29289569.
14. Saka, S. K., Wang, Y., Kishi, J. Y., Zhu, A., Zeng, Y., Xie, W., Kirli, K., Yapp, C., Cicconet, M., Beliveau, B. J., Lapan, S. W., Yin, S., Lin, M., Boyden, E. S., Kaeser, P. S., Pihan, G., Church, G. M., & Yin, P. Immuno-SABER enables highly multiplexed and amplified protein imaging in tissues. Nat. Biotechnol. 37, 1080-1090 (2019) PMID: 31427819.
15. Chung, H., Parkhurst, C. N., Magee, E. M., Phillips, D., Habibi, E., Chen, F., Yeung, B. Z., Waldman, J., Artis, D., & Regev, A. Joint single-cell measurements of nuclear proteins and RNA in vivo. Nat. Methods 2021 1810 18, 1204-1212 (2021) PMID: 34608310.
16. Becht, E., McInnes, L., Healy, J., Dutertre, C. A., Kwok, I. W. H., Ng, L. G., Ginhoux, F., & Newell, E. W. Dimensionality reduction for visualizing single-cell data using UMAP. Nat. Biotechnol. 2018 371 37, 38-44 (2018) PMID: 30531897.

### Example 2: Single Prokaryotic Cell Analysis on Mass Cytometry With ACE

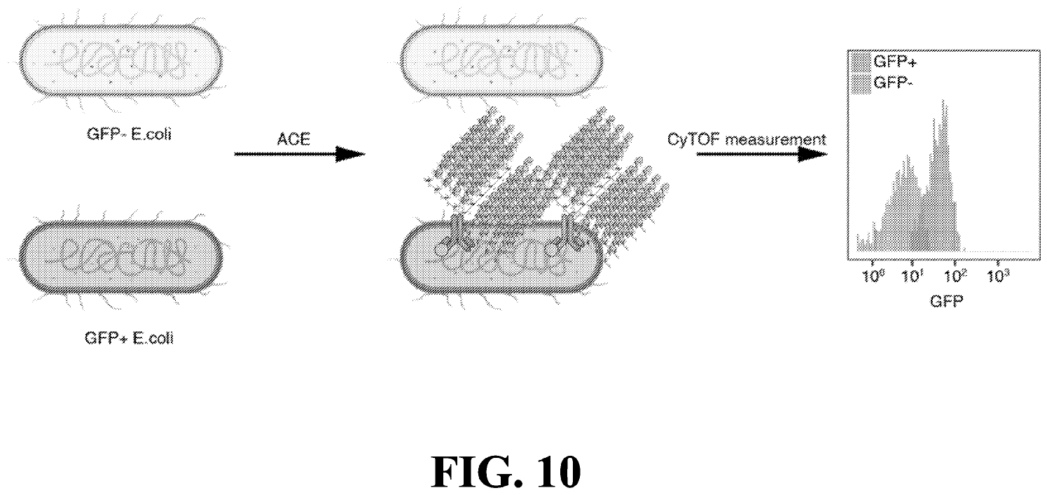

To evaluate the ability of ACE to detect changes in protein abundance in cells as small as bacteria, GFP+ and GFP-E. coli cells were stained using an DNA barcode-conjugated anti-GFP antibody (FIG. 10). With two rounds of branching, protein abundances in microbes could be measured using mass cytometry. 7-fold differences in signal intensity were observed between these two populations, demonstrating that the signal amplification method allows quantitative analysis of even single bacterial cells with mass cytometry.

### Example 3: Primary T Cell Signaling Analysis With ACE

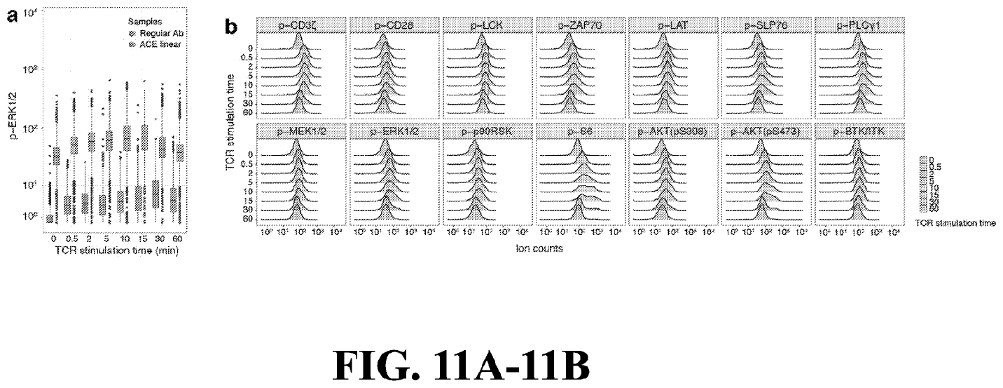

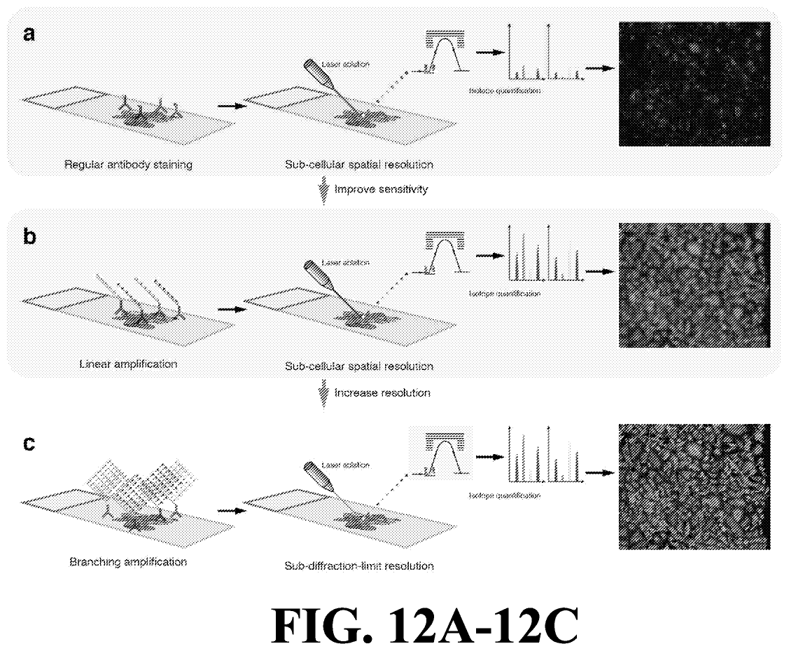

Systematic profiling of TCR signaling networks at single-cell resolution has been technically challenging due to the limited abundance of phospho-proteins in T lymphocytes and their small size. ACE enabled, on average, 17-fold and a maximum of 41-fold signal amplification in a 30-plex TCR signaling profiling panel. In primary human CD4 T cells, p-ERK signals were only slightly above the mass cytometer detection limit using conventionally conjugated antibodies (FIG. 11A). With ACE, p-ERK signals were drastically increased that showed expected trend during a TCR stimulation time course experiment. By analyzing primary human T cells harvested over an 1-hour TCR stimulation time course, ACE revealed differential signaling responses on key TCR signaling mediators, including p-CD3ζ, p-CD28, p-ZAP70/SYK, p-LAT, p-SLP76, p-PLCγ1, p-BTK/ITK, p-MEK1/2, p-ERK1/2, p-p90RSK and p-S6 (FIG. 11B).

### Example 4: Engineering ACE to Enhance the Sensitivity and Resolution in IMC

In imaging mass cytometry (IMC), a UV laser is used to ablate antibody-stained samples spot by spot. A mixed argon and helium stream then transports the ablated materials into a mass cytometer. More than 50 proteins are quantified simultaneously with preserved subcellular level (1 μm²) spatial information (FIG. 12A)^{1,2}. IMC has increasingly been applied to profile microenvironmental features including cell types, functionality, and cell-to-cell interactions in tumor and metabolic diseases^{3,4}. However, as little biomaterials present in every 1 μm² ablation pixel, only high-abundance proteins may be captured to generate detectable signals in the IMC analysis. Due to this limitation, recent IMC-based human breast cancer studies were designed focusing on cell surface epitopes, cytokeratin proteins, and a few cellular functional indicators^{3,5} (FIG. 12A). These proteins typically have high abundance, particularly in the analyzed tissue samples. Such type of spatial and multiplexed epitope analyses plays a significant role in cell type deep classification, and in the subsequent tumor-stromal and immune cell interaction profiling, but lack the sensitivity to study the key phosphorylation sites or transcriptional factors that control or indicate growth, proliferation, survival, and migration, limiting IMC in deciphering cell fate and functional determinants in cancer cells. By applying ACE to amplify IMC signal, a wide selection of low-abundance targets can be detectable on an IMC machine (FIG. 12B).

Using ACE, it is also expected to increase the spatial resolution in IMC analysis. Improving IMC resolution has previously been considered difficult as reducing the laser ablation crater size will generate exponentially fewer ablated ions from each laser pulse (i.e., one pixel in the reconstructed image). A high-strength signal amplification method that allows to multiply the number of conjugated ions on each antibody will be the key to enable a potential high-resolution IMC analysis. Applying two rounds of branching, ACE is expected to increase the averaged metal ion counts by nearly 4000-fold (FIG. 12C). The laser ablation crater size can, therefore, be reduced to a sub-diffraction-limit level of 100 nm in diameter, compared to the currently used 1 μm² laser beam. After the resolution upgrade, ion counts for each laser shot will be collected from a 100 times smaller crater and therefore expected to decrease by 100-fold. However, as compensated by the ultra-high amplification power of ACED, ˜40-fold brighter signals can still be generated, compared to the ion counts from the conventional resolute 1 μm-resolution IMC.

### Example 5: 20-Plex ACE-IMC Analysis on Human Kidney Samples

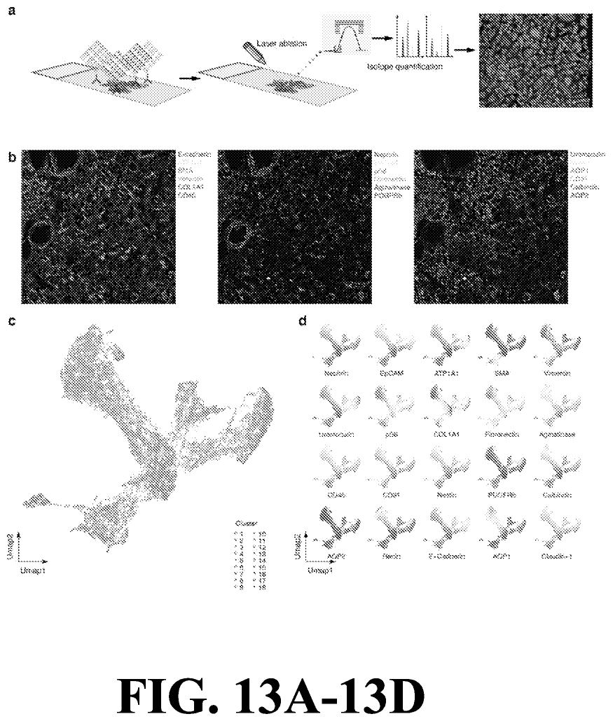

To validate ACE in imaging mass cytometry signal amplification, human kidney sections were stained with 20 ACE oligo conjugated antibodies simultaneously (FIG. 13A). After post-staining fixation, these markers were amplified with linear or branching ACE before analyzing the slides on IMC. 18 of the ACE amplified kidney markers were shown in three overlaid images where each marker was indicated by a specific color (FIG. 13B). Locations of these markers in the kidney tissues were as expected. Different tissue compartments and functional units could be identified in the multiplexed images. Clear signals were observed for low-abundance markers, including the transcriptional factor Nestin, and a phosphorylated protein p-S6. Single-cell segmentation was performed and segmented cells were embedded onto a two-dimensional UMAP plot (FIG. 13C). 18 different cell clusters were identified using Phenograph that was color-coded on the UMAP plot. Each cluster represented a distinct cell type detected using ACE-assisted IMC analysis. Cells types detected in the analysis showed unique protein expression profile that can be only studied with multiplex spatial proteomic analysis powered by ACE (FIG. 13D).

All patents and other publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

1. A method of detecting a target molecule in mass cytometry, the method comprising:
a) contacting a target-binding ligand molecule comprising a conjugated oligonucleotide primer with a cell or tissue sample under conditions permitting binding of the target-binding ligand molecule to its target in the cell or tissue sample;
b) extending the oligonucleotide primer to comprise a concatemer of sequence comprised by the oligonucleotide primer;
c) contacting the extended oligonucleotide primer of step (b) with a labeled probe nucleic acid comprising sequence complementary to sequence repeated in the concatemer, such that a plurality of labeled probe nucleic acid molecules hybridizes to a plurality of sequence repeats in the concatemer;
d) cross-linking the hybridized probe nucleic acid molecules to the extended oligonucleotide primer; and
e) detecting labeled probe using mass cytometry.

2. The method of claim 1, wherein the labeled probe nucleic acid comprises a metal ion label or a fluorophore.

3. (canceled)

4. The method of claim 1, wherein extending the oligonucleotide primer comprises thermal cycling with a single-stranded extender template and a template-dependent nucleic acid polymerase enzyme.

5. (canceled)

6. The method of claim 4, wherein the single-stranded extender template comprises a concatemer of at least two head-to-tail copies of a sequence complementary to the oligonucleotide primer, and a 3′ chain terminator, such that the extender template is not extended by the polymerase.

7. The method of claim 1, wherein extending step (b) comprises a plurality of cycles of extension, thermal strand separation, and cooling to permit single-stranded extender annealing and extension by the polymerase, whereby successive cycles add successive concatemeric repeats to the oligonucleotide primer.

8. The method of claim 1, performed in multiplex with a plurality of different target-binding ligand molecules, orthogonal sets of single-stranded extender templates, and distinguishably labeled orthogonal probe nucleic acids.

9. The method of claim 1, wherein the concatemer comprises a branched concatemer, formed on the target-binding ligand via thermal cycling.

10. The method of claim 1, further comprising the step, before detection step (e), of imaging fluorophore associated with the cell or tissue sample.

11. The method of claim 1, wherein cross-linking the hybridized probe nucleic acid molecules to the extended oligonucleotide primer comprises photo cross-linking.

12.-13. (canceled)

14. The method of claim 1, wherein the target-binding ligand comprises an antibody or antigen-binding fragment thereof.

15. A method of fluorescence-activated cell sorting, the method comprising:
a. providing a sample comprising fixed cells;
b. contacting a target-binding ligand molecule comprising a conjugated oligonucleotide primer with cells in the sample under conditions permitting binding of the target-binding ligand molecule to its target in or on the cells;
c. extending the oligonucleotide primer to comprise a concatemer of sequence comprised by the oligonucleotide primer;
d. contacting the extended oligonucleotide primer of step (c) with a fluorescently labeled probe nucleic acid comprising sequence complementary to sequence repeated in the concatemer, such that a plurality of labeled probe nucleic acid molecules hybridizes to a plurality of sequence repeats in the concatemer;
e. cross-linking the hybridized probe nucleic acid molecules to the extended oligonucleotide primer; and
f. sorting the cells via fluorescence-activated cell sorting.

16.-27. (canceled)

28. A composition comprising a target-binding ligand and a concatemeric oligonucleotide conjugated to the target-binding ligand.

29. The composition of claim 28, wherein the concatemeric oligonucleotide further comprises one or more branched concatemeric oligonucleotides.

30. The composition of claim 28, further comprising a plurality of nucleic acid probe molecules hybridized to concatemeric repeats of the concatemeric oligonucleotide.

31. The composition of claim 30, wherein the nucleic acid probe molecules comprise a cross-linking moiety.

32. (canceled)

33. The composition of claim 31, wherein the nucleic acid probe molecules are cross-linked to the concatemeric repeats.

34. The composition of claim 30, wherein the nucleic acid probe molecules comprise a fluorescent label or a metal ion label.

35. (canceled)

36. The composition of claim 30, wherein the target-binding ligand comprises an antibody or antigen-binding fragment thereof.

37. A cell or tissue sample comprising a composition of claim 30, wherein the target-binding ligand is bound to a target molecule in or on the cell or tissue sample.

38.-52. (canceled)
